(19) United States
(12) Patent Application Publication
(10) Pub. No.: US 2006/0148734 A1
(43) Pub. Date: Jul. 6, 2006

(54) Diaminoacid-aminoacid-polyamine based gemini surfactant compounds

(76) Inventors: Patrick Camilleri, Harlow (GB); Martinus C Feiters, Nijmegen (NL); Anthony John Kirby, Cambridge (GB); Gael Alain Bertrand Ronsin, Cambridge (GB); Roeland Johnnes Maria Nolte, Nijmegen (NL); Cristina Leonor Garcia, Nijmegen (NL)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/508,887

(22) PCT Filed: Mar. 26, 2003
(86) PCT No.: PCT/GB03/01291

§ 371 Date: Jun. 7, 2005

(30) Foreign Application Priority Data
Mar. 27, 2002 (GB) 0207283.3
Jun. 13, 2002 (GB) 0213646.3

(51) Int. Cl. A61K 48/00; C07D 241/04

(52) U.S. Cl. 514/44; 544/399; 564/153

(57) ABSTRACT

Diaminoacid-polyamine:peptide-based gemini compounds are disclosed. The compounds are based on diaminoacid-polyamine or diaminoacid-aminoacid-polyamine backbone with peptide groups and optionally hydrocarboxyl groups linked thereto. Uses of the diaminoacid-polyamine:peptide-based gemini compounds and methods for their production are also disclosed.

This application claims the benefit of UK priority application No. GB0207283.3 filed 27 Mar. 2002 and GB0213646.3 filed 13 Jun. 2002, whose contents are incorporated herein by reference.

This invention relates to newly identified diaminoacid-polyamine:peptide and diaminoacid-aminoacid-polyamine based gemini surfactant compounds, to the use of such compounds and to their production. The invention also relates to the use of the diaminoacid-polyamine:peptide based gemini compounds to facilitate the transfer of compounds into cells for drug delivery.

Surfactants are substances that markedly affect the surface properties of a liquid, even at low concentrations. For example surfactants will significantly reduce surface tension when dissolved in water or aqueous solutions and will reduce interfacial tension between two liquids or a liquid and a solid. This property of surfactant molecules has been widely exploited in industry, particularly in the detergent and oil industries. In the 1970s a new class of surfactant molecule was reported, characterised by two hydrophobic chains with polar heads which are linked by a hydrophobic bridge (Deinega, Y et al., Kolloidn. Zh. 36, 649, 1974). These molecules, which have been termed “gemini” (Menger, F M and Littau, C A, J. Am. Chem. Soc. 113, 1451, 1991), have very desirable properties over their monomeric equivalents. For example they are highly effective in reducing interfacial tension between oil and water based liquids and have a very low critical micelle concentration (Menger, F M and Keiper, J S, Angewandte. Chem. Int. Ed. Engl., 2000, 39, 1906).

Cationic surfactants have been used inter alia for the transfection of polynucleotides into cells in culture, and there are examples of such agents available commercially to scientists involved in genetic technologies (for example the reagent Tfx™-50 for the transfection of eukaryotic cells available from Promega Corp. WI, USA).

The efficient delivery of DNA to cells in vivo, either for gene therapy or for antisense therapy, has been a major goal for some years. Much attention has concentrated on the use of viruses as delivery vehicles, for example adenoviruses for epithelial cells in the respiratory tract with a view to corrective gene therapy for cystic fibrosis (CF). However, despite some evidence of successful gene transfer in CF patients, the adenovirus route remains problematic due to inflammatory side-effects and limited transient expression of the transferred gene. Several alternative methods for in vivo gene delivery have been investigated, including studies using cationic surfactants. Gao, X et al. Gene Ther. 2, 710-722, 1995 demonstrated the feasibility of this approach with a normal human gene for CF transmembrane conductance regulator (CFTR) into the respiratory epithelium of CF mice using amine carrying cationic lipids. This group followed up with a liposomal CF gene therapy trial which, although only partially successful, demons the potential for this approach in humans (Caplen, N J. et al., Nature Medicine, 1, 39-46, 1995). More recently other groups have investigated the potential of other cationic lipids for gene delivery (Miller, A, Angew. Int. Ed. Engl., 37, 1768-1785, 1998), for example cholesterol derivatives (Oudrhiri, N et al. Proc. Natl. Acad. Sci. 94, 1651-1656, 1997). This limited study demonstrated the ability of these cholesterol based compounds to facilitate the transfer of genes into epithelial cells both in vitro and in vivo, thereby lending support to the validity of this general approach.

These studies, and others, show that in this new field of research there is a continuing need to develop novel low-toxicity surfactant molecules to facilitate the effective transfer of polynucleotides into cells both in vitro for transfection in cell-based experimentation and in vivo for gene therapy and antisense treatments. Gemini surfactants based on cysteine (WO99/29712) or on spermine (WO00/77032) or diamine (WO/00/76954) have previously been made. Other examples of gemini surfactants are found in WO/00/27795, WO02/30957 and WO02/50100.

The present invention seeks to overcome the difficulties exhibited by existing compounds.

The invention relates to diaminoacid-polyamine:peptide based gemini compounds having a diaminoacid-polyamine or a diaminoacid-aminoacid-polyamine backbone and conforming to the general structure of formula (I): 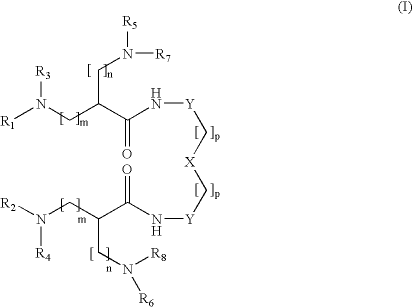
where:
m=0 to 6;
n=0 to 7;
p=0 to 6; and where
X=a bond, CH₂, (CH₂)₂, NH(CH₂)qNH where q=2 to 6, or 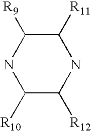
where R₉ to R₁₂, which can be the same or different, are selected from H, O or CᵣH₂ᵣ₊₁, where r=0 to 6 with the proviso that when R₉ and R₁₂ are O, or when R₉ and R₁₁ are O, then R₁₀ and R₁₁ or R₁₀ and R₁₂, respectively, are H; and where
Y=a bond, CH₂, 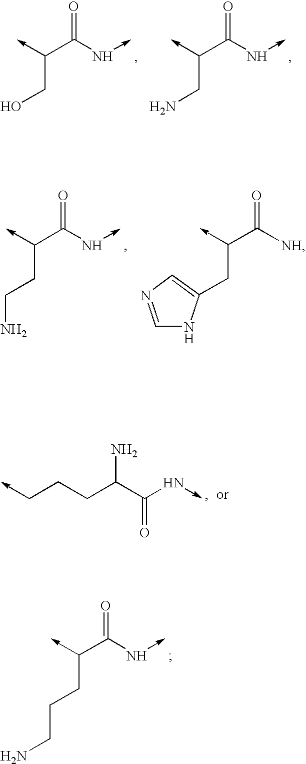
and where R₃, R₄, R₅, R₆, R₇ and R₈ are hydrogen and R₁ and R₂ are saturated or unsated hydrocarboxyl groups having up to 24 carbon atoms and linked to the diaminoacid-polyamine backbone by an amide bond; or
where R₃, R₄, R₅ and R₆ are hydrogen, R₁ and R₂ are saturated or unsaturated hydrocarboxyl groups having up to 24 carbon atoms and linked to the diaminoacid-polyamine backbone by an amide bond, and where R₇ and R₈, which may be the same or different, are peptide groups formed from one or more amino acids linked together by amide (CONH) bonds and further linked to the diaminoacid-polyamine backbone by amide bonds, in a linear or branched manner, having the general formula (II): 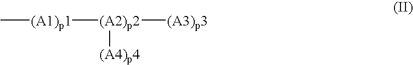
where the values for p1 and p2, which may be the same or different are from 0 to 5, preferably 1; and the values for p3 and p4, which may be the same or different, are from 0 to 5, preferably 0; A1, A3 and A4, which may be the same or different, is an amino acid selected from serine, lysine, ornithine, threonine, histidine, cysteine, arginine and tyrosine; and
A2 is an amino acid selected from lysine, ornithine and histidine;
   or
a salt, preferably a pharmaceutically acceptable salt thereof.

Preferably, the compound is symmetrical, that is R₁ and R₂ are the same as each other, R₃ and R₄ are the same as each other, R₅ and R₆ are the same as each other, R₇ and R₈ are the same as each other.

In a preferred embodiment A1 is lysine, serine or threonine, preferably lysine. Preferably A3 and A4 are lysine, ornithine, histidine or arginine.

In a further preferred embodiment the hydrocarboxyl group is selected from:
—C(O)(CH₂)₁₀CH₃
—C(O)(CH₂)₁₂CH₃
—C(O)(CH₂)₁₄CH₃
—C(O)(CH₂)₁₆CH₃
—C(O)(CH₂)₁₈CH₃
—C(O)(CH₂)₂₀CH₃
—C(O)(CH₂)₇CH═CH(CH₂)₅CH₃ natural mixture
—C(O)(CH₂)₇CH═CH(CH₂)₇CH₃ natural mixture
—C(O)(CH₂)₇CH═CH(CH₂)₅CH₃ Cis
—C(O)(CH₂)₇CH═CH(CH₂)₇CH₃ Cis
—C(O)(CH₂)₇CH═CH(CH₂)₅CH₃ Trans
—C(O)(CH₂)₇CH═CH(CH₂)₇CH₃ Trans
—C(O)(CH₂)₇CH═CHCH₂CH═CH(CH₂)₄CH₃
—C(O)(CH₂)₇(CH═CHCH₂)₃CH₃
—C(O)(CH₂)₃CH═CH(CH₂CH═CH)₃(CH₂)₄CH₃
—C(O)(CH₂)₇CHCH(CH₂)₇CH₃
—C(O)(CHCHOH(CH₂)₂CH₃ or
—C(O)(CH₂)₂₂CH₃.
Most preferably the hydrocarboxyl group is selected from (CH₂)₇ CH═CH(CH₂)₇CH₃ natural mixture, (CH₂)₇CH═CH(CH₂)₇CH₃ Cis and (CH₂)₇ CH═CH(CH₂)₇CH₃ Trans.

In a preferred embodiment m is 0, n is 2 to 4, X is (CH₂) or (CH₂)₂, Y is a bond and p is 0 to 4.

In a further preferred embodiment m is 0, n is 2 to 4, X is NH(CH₂)qNH, where q is 2 to 5, Y is a bond and p is 2 to 5.

In another preferred embodiment m is 0, n is 2 to 4, X is 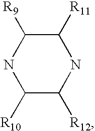
where R₉, R₁₀, R₁₁ and R₁₂ are all H, Y is a bond and p is 2 to 5.

In a still further preferred embodiment m is 0, n is 2 to 4, X is (CH₂) or (CH₂)₂, p is 0 to 4 and Y is

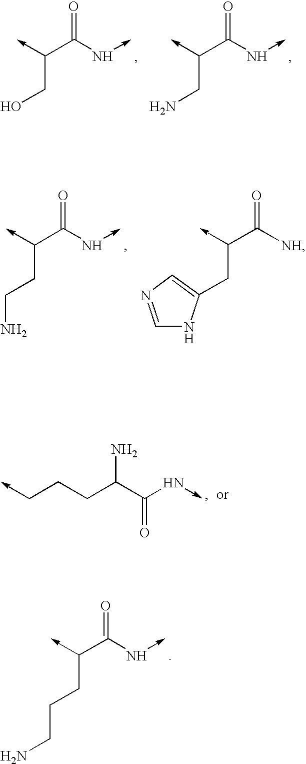

In a yet further preferred embodiment m is 0, n is 2 to 4, X is NH(CH₂)qNH where q is 2 to 5, p is 2 to 5 and Y is

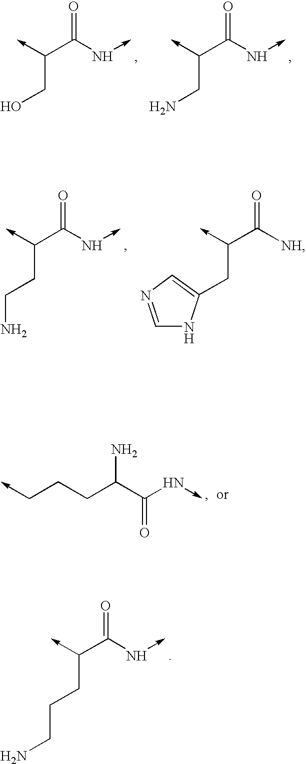

In a yet still further preferred embodiment m is 0, n is 2 to 4, X is 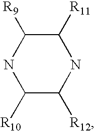
where R₉, R₁₀, R₁₁ and R₁₂ are all H, p is 2 to 5 and Y is

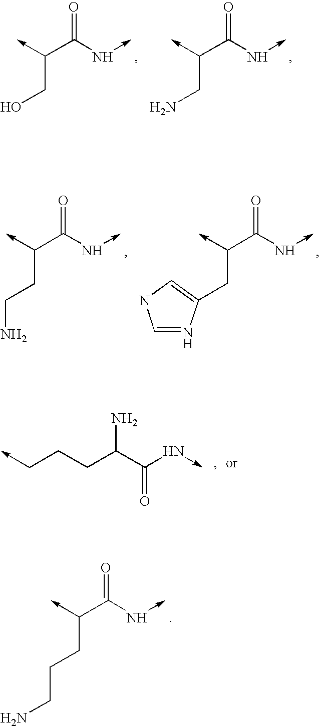

A further preferred embodiment is where X is 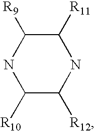
Y is a bond, p is 1 to 6 and n is 1 to 7.

Compounds of the present invention may be prepared from readily available starting materials using synthetic peptide chemistry well known to the skilled person. The scheme shown in FIG. 1 shows a general scheme for the synthesis of the compounds of the invention wherein the hydrocarboxyl groups are linked to the α-amino group of a diaminoacid further linked to a polyamine backbone moiety by amide bonds, the scheme shown in FIG. 2 shows a general scheme for the synthesis of the compounds of the invention wherein the hydrocarboxyl groups are linked to the terminal amino group of a diaminoacid further linked to a polyamine backbone moiety by amide bonds and the scheme shown in FIG. 3 shows a general scheme for the synthesis of diaminoacid-aminoacid-polyamine:peptide based gemini compounds wherein an aminoacid is linked by an amide bond to the amino group (α or terminal) of a diaminoacid further linked to a polyamine moiety by an amide bond.

Another aspect of the invention relates to methods for using the diaminoacid-polyamine:peptide based gemini compounds. Such uses include facilitating the transfer of oligonucleotides and polynucleotides into cells for antisense, gene therapy and genetic immunisation (for the generation of antibodies) in whole organisms. Other uses include employing the compounds of the invention to facilitate the transfection of polynucleotides into cells in culture when such transfer is required, in, for example, gene expression studies and antisense control experiments among others. Protocols for the preparation of such polynucleotides and antisense molecules are well known in the art (for example Sambrook et al., Molecular Cloning: A Laboratory Manual, 2nd Ed., Cold Spring Harbor Laboratory Press, Cold Spring Harbor, N.Y. (1989), Cohen, J S ed. Oligodeoxynucleotides as Antisense Inhibitors of Gene Expression, CRC Press, Boca Raton, Fla. (1989)). The polynucleotides can be mixed with the compounds, added to the cells and incubated to allow polynucleotide uptake. After further incubation the cells can be assayed for the phenotypic trait afforded by the transfected DNA, or the levels of mRNA expressed from said DNA can be determined by Northern blotting or by using PCR-based quantitation methods for example the Taqman® method (Perkin Elmer, Connecticut, USA). Compounds of the invention offer a significant improvement, typically between 3 and 6 fold, in the efficiency of cellular uptake of DNA in cells in culture, compared with compounds in the previous art. In the transfection protocol, the gemini compound may be used in combination with one or more supplements to increase the efficiency of transfection. Such supplements may be selected from, for example:
(i) a neutral carrier, for example dioleyl phosphatidylethanolamine (DOPE) (Farhood, H., et al (1985) Biochim Biophys. Acta, 1235-1289);
(ii) a complexing reagent, for example the commercially available PLUS reagent (Life Technologies Inc. Maryland, USA) or peptides, such as polylysine or polyornithine peptides or peptides comprising primarily, but not exclusively, basic amino acids such as lysine, ornithine and/or arginine. The list above is not intended to be exhaustive and other supplements that increase the efficiency of transfection are taken to fall within the scope of the invention.

In still another aspect, the invention relates to the transfer of genetic material in gene therapy using the compounds of the invention. For example the skilled person can develop gene delivery methodologies for use in gene therapy, involving the use of gemini surfactant compounds of the present invention, using protocols that are well known in the art. For example the use of surfactants for delivery of gene transfer vectors to the lung is reviewed in Weiss, D J (2002) Molecular Therapy 6(2) pp 148 to 152.

Yet another aspect of the invention relates to methods to effect the delivery of non-nucleotide based drug compounds into cells in vitro and in vivo using the compounds of the invention.

The following definitions are provided to facilitate understanding of certain terms used frequently herein.

“Amino acid” refers to dipolar ions (zwitterions) of the form ⁺H₃NCH(R)CO₂⁻. They are differentiated by the nature of the group R, and when R is different from hydrogen can also be asymmetric, forming D and L families. There are 20 naturally occurring amino acids where the R group can be, for example, non-polar (e.g. alanine, leucine, phenylalanine) or polar (e.g. glutamic acid, histidine, arginine and lysine). In the case of un-natural amino acids R can be any other group which is not found in the amino acids found in nature.

“Polynucleotide” generally refers to any polyribonucleotide or polydeoxribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNA's or RNA's containing one or more modified bases and DNA's or RNA's with backbones modified for stability or for other reasons. “Modified” bases include, for example, tritylated bases and unusual bases such as inosine. A variety of modifications have been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Transfection” refers to the introduction of polynucleotides into cells in culture using methods involving the modification of the cell membrane either by chemical or physical means. Such methods are described in, for example, Sambrook et al., MOLECULAR CLONING: A LABORATORY MANUAL, 2nd Ed., Cold Spring Harbor Laboratory Press, Cold Spring Harbor, N.Y. (1989). The polynucleotides may be linear or circular, single-stranded or double-stranded and may include elements controlling replication of the polynucleotide or expression of homologous or heterologous genes which may comprise part of the polynucleotide.

The invention will now be described by way of the following examples.

### EXAMPLES

### Example 1

### CR-110

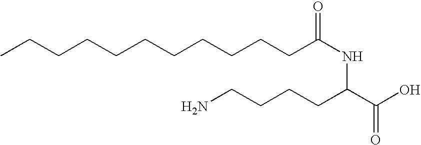

To a solution of H-Lys(Boc)-OH (5.02 g, 20.4 mmol) and 20.5 mL of NaOH 1M in 85 Ml of water-acetone (1:2 v/v) cooled at 0° C. was added dropwise 4.43 g (20.3 mmol) of dodecyl chloride and NaOH aq. 1M alternatively to maintain the pH over 9. After addition keep 10 minutes more stirring at 0° C. HCl 10% was added until pH 2. Filter the solid and wash with water until pH 7. Dry over P₂O₅. The solid is chromatographied on silica with CHCl₃—MeOH to yield 46% of compound CR-110 as a white solid. α_{D}²⁰−1.0 (c 1.48, MeOH); IR(KBr)vₘₐₓ 3347, 2921, 2851, 1717, 1681, 1521 cm⁻¹; ¹H NMR (300 MHz, CD₃OD) 4.26 (dd, 1H, J=4.77, 8.92 Hz, CH—COOH), 2.94 (t, 2H, J=6.7 Hz, CH₂N), 2.16 (t, 2H, J=7.4 Hz, CH₂CON), 1.78-1.74 (m, 1H, HCH—CH(COOH), 1.63-1.45 (m, 7H, HCH—CH(COOH), CH₂CH₂N and CH₂CH₂CON), 1.35 (s, 9H, (CH₃)₃C), 1.22 (s, 16H, CH₃(CH₂)₈), 0.82 (t, 3H, J=6.8 Hz, CH₃); ¹³C (75 MHz, CD₃OD) 176.32 C(O)NCH₂), 175.45 (COOH), 158.42 (C(O)NO), 79.73 (C(CH₃)₃), 54.84 (CH), 41.16 (CH₂N), 36.97, 33.04, 32.64, 30.74-29.99 (CH₂), 28.83 (CH₃), 26.98, 24.26, 23.71 (CH₂), 14.48 (CH₃).

### Example 2

### CR-116

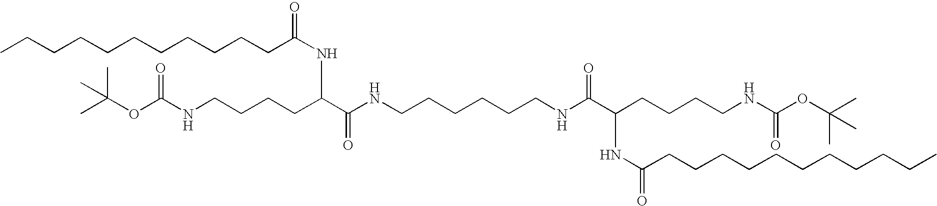

To a solution of 2.4 g (5.6 mmol) of CR-110 in THF at −20° C. were added Et₃N (0.78 mL, 5.6 mmol) and EtOCOCl (0.55 mL, 5.6 mmol). The reaction was stirring at this temperature for 30 minutes and 246 mg (2.8 mmol) of 1,4-diaminobutane were added, after 1 hour more stirring at −20° C. the reaction mixture was allowed to warm at room temperature and stirred overnight. Remove the solvent in vacuum, the residue was dissolved in CHCl₃ and washed with NaHCO₃ aq. saturated and brine and dried over MgSO4 anh. The obtained residue was chromatographied to give compound CR-116 (50%) as a white solid: α_{D}²⁰−10.06 (c 1.51, MeOH); IR(KBr)vₘₐₓ 3415-3307, 2920, 2851, 1688, 1637, 1515 cm⁻¹; ¹H NMR (300 MHz, CD₃OD) 4.17 (dd, 1H, J=5.5, 8.5 Hz, CH—COOH), 3.12 (m, 2H, CH₂N), 2.96 (q, 2H, J=6.4 Hz, CH₂N), 2.17 (t, 2H, J=7.4 Hz, CH₂C(O)N), 1.69-1.64 (m, 1H, HCH—HC(COOH), 1.58-1.42 (m, 5H, HCH—HC(COOH), CH₂CH₂CO, CH₂CH₂N), 1.36 (s, 9H, (CH₃)₃C), 1.22 (s, 16H, CH₃(CH₂)₈CH₂), 0.88 (t, 2H, J=6.8 Hz, CH₃); ¹³C (75 MHz, CD₃OD) 176.26, 174.46 C(O)NCH₂), 158.42 (OC(O)N), 79.93 (C(CH₃)₃), 54.82 (CH) 41.11 and 39.96 (CH₂N), 36.89, 33.09, 32.92, 30.77-30.38 (CH₂), 28.85 (CH₃), 27.63, 26.94, 24.28, 23.74(CH₂), 14.48 (CH₃).

### Example 3

### CR-117: GSN11

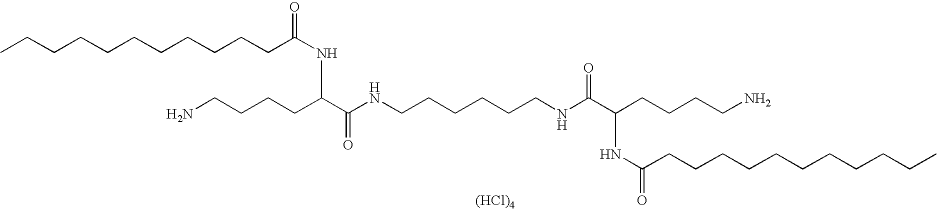

1.2299 g (1.35 mmol) of CR-116 were treated with EtOAc 4 M for 45 minutes. The solid was filtered and recrystalized from MeOH and EtOAc added to obtain the compound CR-117 as a white solid (49%): α_{D}²⁰−13.98 (c 1.76, MeOH); IR(KBr)vₘₐₓ 3422, 3298, 3089, 2920, 2851, 1638 cm⁻¹; ¹H NMR (300 MHz, CD₃OD) 4.20 (dd, 1H, J=5.6, 8.4 Hz, CH—COOH), 3.12 (m, 2H, CH₂N), 2.84 (t, 2H, J=6.4 Hz, CH₂N), 2.18 (t, 2H, J=7.6 Hz, CH₂C(O)N), 1.74-1.72 (m, 1H, HCH—CH(COOH), 1.69-1.34 (m, 5H, HCH—CH(COOH)+CH₂CH₂CO+CH₂CH₂N), 1.22 (s, 16H, CH₃(CH₂)₈CH₂), 0.82 (t, 2H, J=6.8 Hz, CH₃); ¹³C (75 MHz; CD3OD) 176.39, 174.22 C(O)NCH₂), 54.59 (CH), 40.55, 39.99 (CH₂N), 33.08, 32.57, 30.76-30.41(CH₂), 28.23, 27.61, 26.93 (CH₂), 14.44 (CH₃); C₄₀H₈₀Cl₂N₆O₄.H₂O 778.56 calc C 60.94%, H 10.36%, N 10.65% found C 60.88%, H10.22%, N 10.08%

### Example 4

### RG 00/781

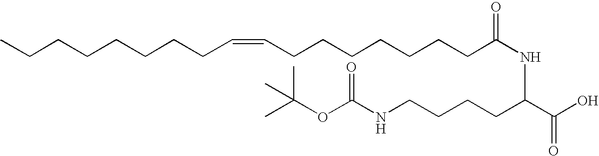

To a solution of N-ε-(tertbutoxycarbonyl)-L-lysine (1.24 g, 5.03 mmol) in THF (140 mL) were added successively a solution of K₂CO₃ (0.75 g, 5.43 mmol, 1.08 eq.) in water (20 mL) and oleoyl succinimidate (1.92 g, 5.06 mmol, 1 eq.). The reaction was stirred at RT for 20 h and most of THF was evaporated. Water and CHCl₃ (30 mL each) were added and the organic layer was separated. The aqueous layer was acidified to pH 2 and extracted twice with CHCl₃ (2×30 mL). The organic layer was washed with water and brine (20 mL each), dried (Na₂SO₄), filtered and evaporated to give an oil. Yield: 2.46 g (4.82 mmol, 96%). ¹H NMR (400 MHz, d₆-DMSO): δ 12.4 (m, 1 H^{OH}), 7.92 (d, 1 H, J=7.8, HN^{α}), 6.70 (t, 1 H, J=6.0, HN^{ε}), 5.29 (m, 2 CH^{9,10}), 4.10 (dt, 1 H, J=5.0, 8.9, CH^{α}), 2.85 (q, 2 H, J=6.2, CH₂^{ε}), 2.07 (dt, 2 H, J=2.2, 7.0, CH₂²), 1.95 (q, 4 H, J=6.0, CH₂^{8,11}), 1.62 (m, 1 H, CH^{β}), 1.51 (m, 1 H, CH^{β}), 1.45 (m, 2 H, CH₂³), 1.33 (s, 9 H, C(CH₃)₃), 1.2 (m, 26 H, 2 CH₂^{γ,δ} and 10 CH₂ oleoyl), 0.82 (t, J=6.4, 3 H, CH₃¹⁸).

### Example 5

### RG 00/366

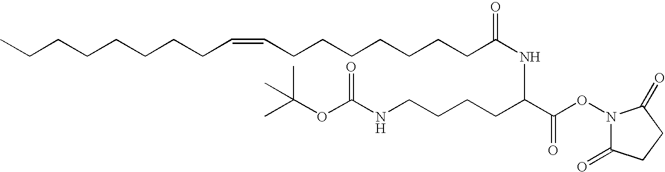

To a solution of N-α-oleoyl-N-ε-(tert-butyloxycarbonyl)-L-Lysine (1.80 g, 3.52 mmol) in THF (80 mL) were added successively N-hydroxysuccinimde (0.41 g, 3.56 mmol, 1.01 eq.) and DCC (0.73 g, 3.54 mmol, 1.01 eq.). The reaction was stirred for 16 h at RT. The precipitate was filtered and washed with EtOAc (30 mL). The filtrate was concentrated and redissolved in EtOAc and filtered again. The residue was dissolved in CHCl₃ and precipitated with Et₂O to give N-α-oleate-N-ε-(tert-butyloxycarbonyl)-L-Lysinyl succinimidate as a white solid. Yield: 1.98 g (93%). NMR ¹H (400 MHz, CDCl₃): δ 6.11 (m, 1 H, HN^{α}), 5.38 (m, 2 H, H^{9,10}), 4.94 (m, 1 H, CH^{α}), 4.65 (m, 1 H, HN^{ε}), 3.12 (m, 2 H, CH₂^{ε}), 2.79 (s, 4 H, 2 CH₂^{Su}), 2.20 (t, J=6.1, 2 H, CH₂²), 2.00 (m, 5 H, CH^{β} and 2 CH₂^{8,11}), 1.84 (m, 1 H, CH^{β}), 1.63 (m, 2 H, CH₂³), 1.48 (m, 4 H, 2 CH₂^{γ,δ}), 1.37 (s, 9 H, 3 CH₃), 1.27 (m, 20 H, 10 CH₂ oleoyl), 0.83 (t, J=6.3 Hz, 3 H, CH₃¹⁸).

### Example 6

### RG 00/250

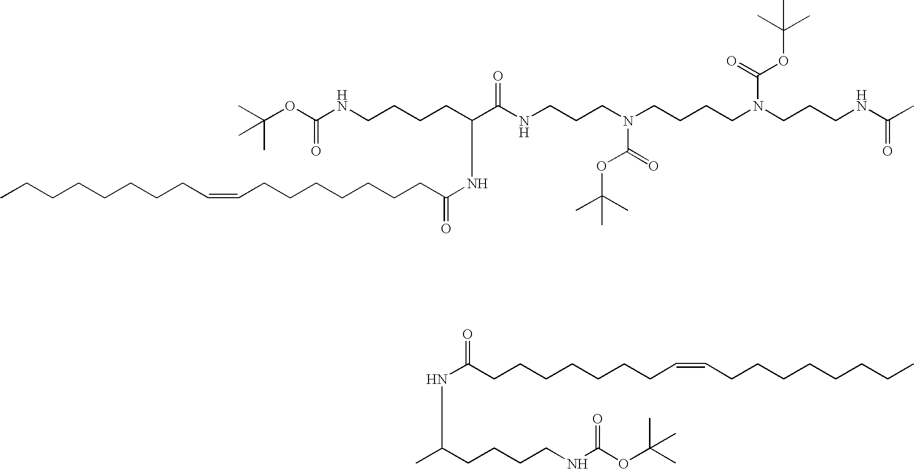

To a solution of N⁴,N⁹-bis-tert-butyloxycarbonyl)-1,12-diamino-4,9-diazadodecane (629 mg, 1.0 mmol) in THF (80 mL) and K₂CO₃ (0.29 g, 2.1 mmol, 2.1 eq.) in water (10 mL) was added a solution of N-α-oleoyl-N-ε-(tert-butyloxycarbonyl)-L-lysinyl succinimidate (1246 mg, 2.05 mmol, 2.05 eq.). The reaction was stirred overnight at RT. Most of the THF was evaporated and water (30 mL) was added. The aqueous layer was extracted with CHCl₃ (2×50 mL). The organic layer was washed with water, 0.1 M HCl, water and brine (20 mL each), dried (Na₂SO₄), filtered, evaporated and purified by column chromatography on SiO₂ (CHCl₃/MeOH: 95/5, Rf=0.30) to give an oil. Yield: 1060 mg (0.76 mmol, 76%). ¹H NMR (400 MHz, CDCl₃): δ 7.30 (bs, 2 H, 2 NHC^{1′}), 6.33 (bs, 2 H, 2 NH^{α}), 5.31 (m, 4 H, 2 CH^{9,10}), 4.71 (bs, 2 H, 2 NH^{ε}), 4.41 (m, 2 H, 2 CH^{α}), 3.18 (m, 12 H, 2 CH₂^{1′}, 2 CH₂^{3′}), 3.08 (m, 4 H, 2 CH₂^{3′} and 2 CH₂^{ε}), 2.18 (t, 4 H, J=6.8, 2 CH₂²), 1.98 (m, 8 H, 2 CH₂^{8,11}), 1.90 (m, 2 H, 2 CH^{β}), 1.79 (m, 2 H, 2 CH^{β}), 1.60 (m 10 H, 2 CH₂^{2′}, 2 CH₂^{γ} and 2 CH₂³), 1.45 (m, 26 H, 2 CH₂^{δ}, 2 CH₂^{5′} and 2 C(CH₃)₃), 1.40 (s, 18 H, 2 C(CH₃)₃), 1.25 (m, 40 H, 2×10 CH₂^{Tail}), 0.86 (m, 6 H, J=6.6, 2 CH₃¹⁸). ¹³C NMR (100 MHz, CDCl₃): δ 171.1, 174.3, 155.6, 155.7, 129.5, 129.3, 79.4, 78.5, 76.8, 52.4, 48.6, 46.4, 39.6, 36.1, 33.5, 31.4, 29.3, 29.2, 29.0, 28.8, 28.7, 28.0, 26.7, 25.3, 24.5, 22.2, 22.1, 13.7.

### Example 7

### RG 00/267: GSC 102

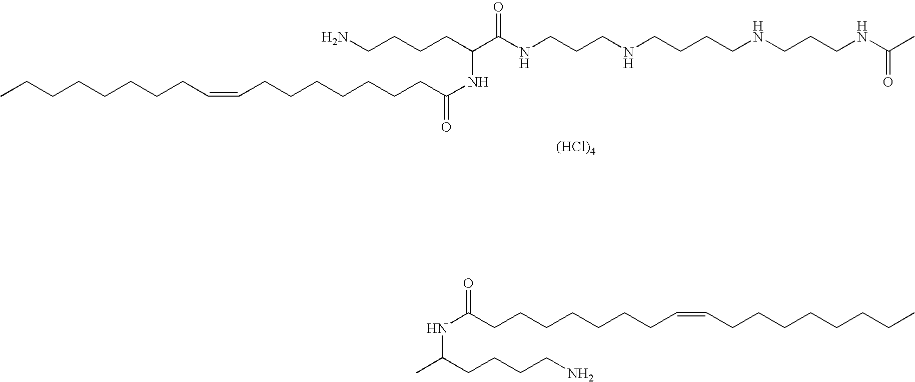

To a solution of RG 00/250 (1.04 g, 0.75 mmol) in MOH (20 mL) was added concentrated HCl (10 mL) and the reaction was stirred at RT for 2 h. The solvent were then removed and the residue redissolved in water (80 mL), filtered on a frit and evaporated again. The residue was redissolved in a minimum volume of methanol and precipitated with Et₂O to give, after filtration, a pale yellow solid. Yield: 0.734 g (0.65 mmol, 86%). ¹H NMR (400 MHz, d₆-DMSO): δ 9.02 (m, 4 H, 2 NH), 8.16 (t, 2 H, J=6.0, 2 NHC^{1′}), 7.98 (s, 6 H, 2 N^{α}H and 2 N^{ε}H₂), 5.29 (m, 4 H, 2 CH^{9,10}), 4.10 (q, 2 H, J=7, 2 CH^{α}), 3.10 (hp, 4 H, J=6.4, 2 CH₂^{1′}), 2.85 (m, 8 H, 2 CH₂^{3′} and 2 CH₂^{4′}), 2.71 (m, 4 H, 2 CH₂^{ε}), 2.10 (AB, 4 H, J=6.4, 2 CH₂²), 1.95 (m, 8 H, 2 CH₂^{8,11}), 1.76 (m, 4 H, 2 CH₂^{2′}), 1.68 (m, 4 H, 2 CH₂^{5′}), 1.65-1.42 (m, 12 H, 2 CH₂^{ε}, 2 CH₂^{δ} and 2 CH₂³), 1.25 (m, 44 H, 10 CH₂^{O1}and 2 CH₂^{γ}), 0.83 (t, 6 H, 2 CH₃¹⁸). MS (+ES): 999.8 [M+Na].

### Example 8

### RG00/371

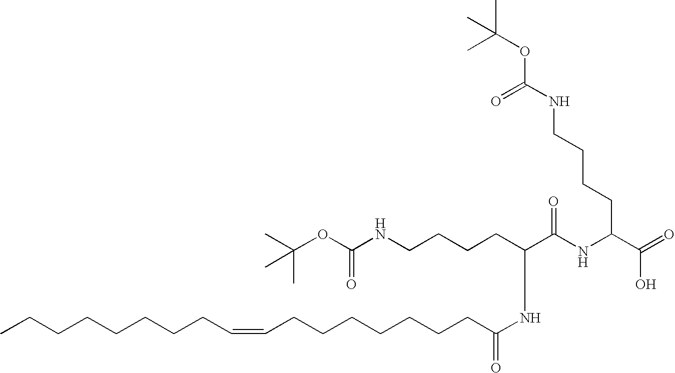

To a solution of N-α-oleoyl-N-ε-(tert-butyloxycarbonyl)-L-lysine (900 mg, 1.48 mmol) in THF (60 mL) were added successively a solution of potassium carbonate (225 mg, 1.63 mmol, 1.1 eq.) in water (6 mL) and N-ε-(tert-butyloxycarbonyl)-L-lysine (365 mg, 1.49 mmol, 1 eq.). The solution was then stirred for 16 h at RT. Most of THF was evaporated and pH of the aqueous solution was adjust to 2 and extra with CHCl₃ (2×80 mL). The organic layer was washed with water (50 mL) and brine (40 mL), dried (Na₂SO₄), filtered and evaporated. The oil obtained was then dissolved in a small quantity of CHCl₃ and Et₂O was added. The white solid was then collected. Yield: 1008 mg (1.46 mmol, 99%). ¹H NMR (400 MHz, CDCl₃): δ 12.60 (m, 1 H, COOH), 8.55 (m, 1 H, NH), 7.10 (m, 1 H, 1 NH), 6.70 (m, 1 H, 1 NH), 5.32 (m, 2 H, CH^{9,10}), 4.80 (m, 1 H, NH), 4.51 (m, 2 H, 2 CH^{α}), 3.08 (m, 4 H, 2 CH₂^{ε}), 2.20 (t, 2 H, J=7.0, 2 CH₂²), 1.99 (m, 4 H, CH₂^{8,11}), 1.60 (m, 4 H, 2 CH₂^{β}), 1.50-1.20 (m, 44 H), 0.87 (t, 3 H, J=6.8, CH₃¹⁸).

### Example 9

### RG 00/376

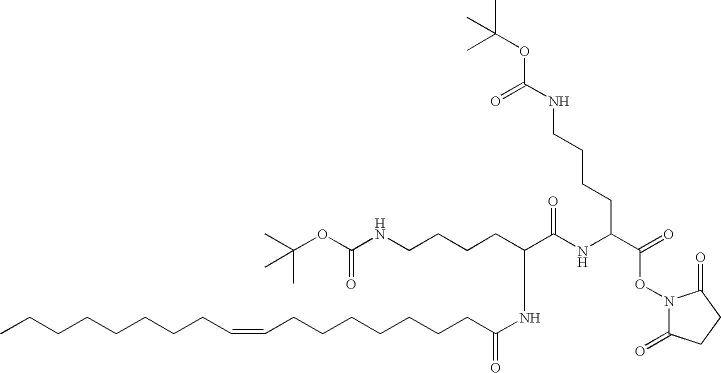

To a solution of N-α-(N-α-Oleoyl-N-ε-(tert-butyloxycarbonyl)-L-lysyl)-N-ε-(tert-butyloxycarbonyl))-L-lysine (1008 mg, 1.46 mmol) in THF (40 mL) was added N-hydroxysuccinimide (177 mg, 1.49 mmol, 1.02 eq.) and DCC (311 mg, 1.50 mmol, 1.03 eq.). The reaction was stirred overnight at RT and the DCU was then filtered and washed with EtOAc. The solvent was then removed and the residue redissolved in EtOAc, the DCU filtered again and after evaporation a white solid was isolated. Yield: 1147 mg (1.36 mmol, 93%).

### Example 10

### RG 00/384

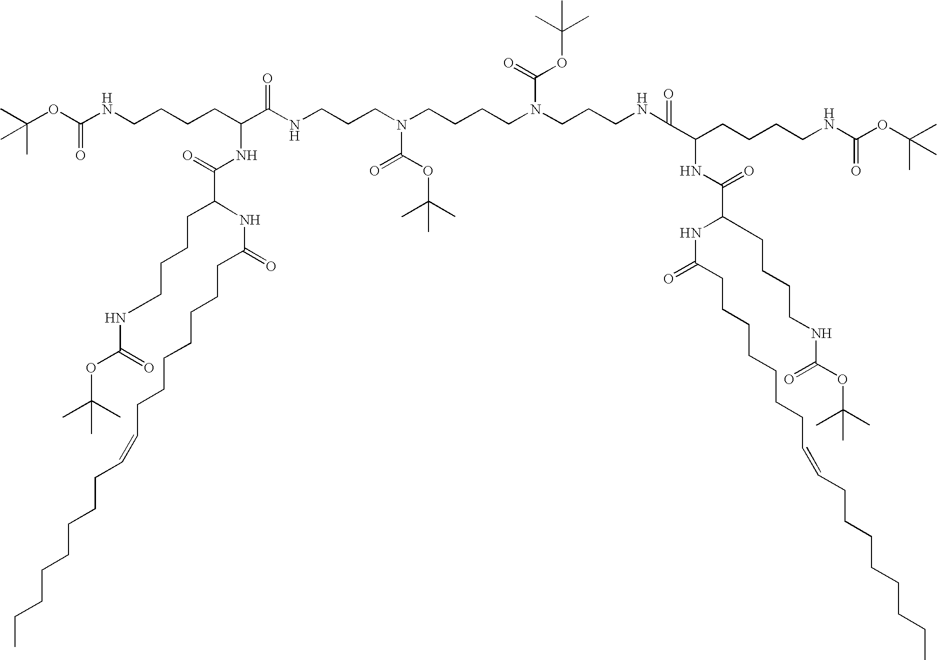

To a solution of N⁴,N⁹-bis-tert-butyloxycarbonyl)-1,12-diamino-4,9-diazadodecane (241 mg, 0.36 mmol) in THF (60 mL) and K₂CO₃ (0.10 g, 0.73 mmol, 2.1 eq.) in water (8 mL) was added a solution of N-α-(N-α-oleoyl-N-ε-(tert-butyloxycarbonyl)-L-Lysyl)-N-ε-tert-butyloxycarbonyl))-L-lysyl succinimidate (600 mg, 0.72 mmol, 2.0 eq.) in THF (10 mL). The reaction was stirred overnight at RT. Most of the THF was evaporated and water (30 mL) was added. The aqueous layer was extracted with CHCl₃ (2×60 mL). The organic layer was washed with water, 0.1 M HCl, water and brine (20 mL each), dried (Na₂SO₄), filtered, evaporated and purified on SiO₂ (CHCl₃/MeOH: 9/1, Rf=0.27) to give a white solid. Yield: 497 mg (0.27 mmol, 75%). ¹H NMR (400 MHz, CDCl₃): δ 8.40 (m, 2 H,2 NH), 6.90 (m, 2 H, 2 NH), 6.40 (m, 2 H, 2 NH), 5.33 (m, 4H, 2 CH^{9,10}), 4.85 (m, 4 H, 4 N^{ε}H), 4.40 (m, 4 H, 2×2 CH^{α}), 3.28-3.02 (m, 20 H, 2×2 CH₂^{ε}, 2 CH₂^{1′}, 2 CH₂^{3′} and 2 CH₂^{4′}), 2.22 (m, 4 H, 2 CH₂²), 1.99 (m, 8 H, 2 CH₂^{8,11}), 1.80 (m, 4 H, 2 CH₂^{β}), 1.72-1.25 (m, 126 H), 0.83 (t, 6 H, J=6.8, 2 CH₃¹⁸).

### Example 11

### RG 00/404

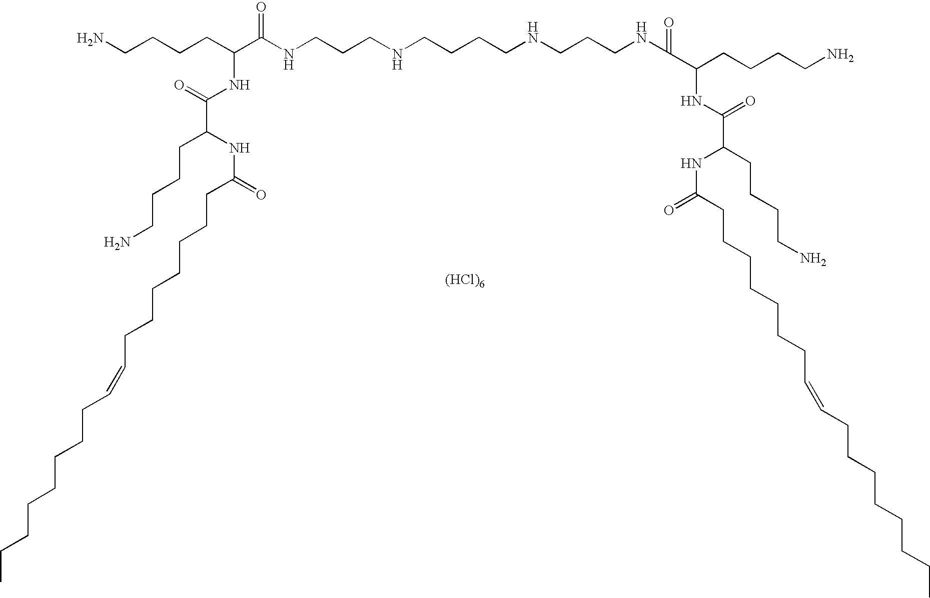

To a solution of RG 00/384 (470 mg, 0.255 mmol) in MeOH (10 mL) was added concentrated HCl (10 mL) and the reaction was stirred at RT for 1 h. The solvents were removed under vacuum and the residue redissolved into water (80 mL), filtered and evaporated again. The residual oil was dissolved in MeOH and precipitated with Et₂O to give a yellow powder. Yield: 284 mg (0.194 mmol, 76%). ¹H NMR (400 MHz, d₆-DMSO): δ 9.10 (m, 4 H, 2 NH₂⁺), 8.18 (m, 4 H, 4 NHC), 8.10-7.98 (m, 16 H 2×2 N^{α}H and 2×2 N^{ε}H₃⁺), 5.29 (m, 4 H, 2 CH^{9,10}), 4.18 (m, 2 H, CH^{α}), 4.11 (m, 2 H, 2 CH^{α}), 3.10 (m, 4 H, 2 CH₂^{1′}), 2.85 (m, 8 H, 2 CH₂^{3′} and 2 CH₂^{4′}), 2.71 (m, 8 H, 2×2 CH₂^{ε}), 2.10 (m, 4 H, 2 CH₂²),1.95 (m, 8 H, 2 CH₂^{8,11}), 1.80-1.39 (m, 28 H), 1.25 (m, 48 H, 10 CH₂^{O1} and 2×2 CH₂^{γ}), 0.83 (t, 6 H, J=6.8, 2 CH₃¹⁸).

### Example 12

### RG 00/278

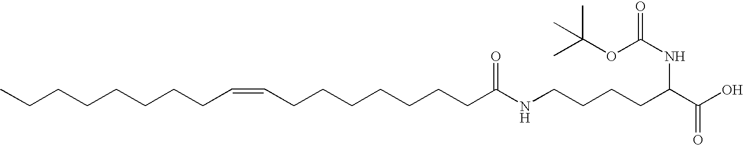

To a solution of N-α-(tert-butyloxycarbonyl)-L-Lysine (779 mg, 3.16 mmol) in THF (80 mL) were added successively a solution of potassium carbonate (0.524 g, 3.79 mmol, 1.2 eq.) in water (10 mL) and oleoyl succinimidate (1.20 g, 3.16 mmol, 1 eq.). The reaction is stirred overnight at room temperature. Most of THF was evaporated and water (40 mL) was added. The aqueous layer was acidified to pH 2 and extracted with CHCl₃ (3×60 mL). The combined organic layers were washed with water (30 mL) and brine (40 mL), dried over sodium sulphate, filtered and evaporated to give N-α-(tert-butyloxycarbonyl)-N-ε-oleoyl-L-lysine as a colourless oil. Yield: 1.31 g (2.56 mmol, 81%). ¹H NMR (400 MHz, CDCl₃): d 5.78 (t, 1 H, J=8.0, NH^{ε}), 5.33 (m, 2 H, CH^{9,10}), 5.27 (d, 1 H, J=7.8, NH^{α}), 4.27 (m, 1 H, CH^{α}), 3.24 (q, 2 H, J=8.0, CH₂^{ε}), 2.26 (t, 2 H, J=6.8, CH₂²), 1.98 (m, 4 H, CH₂^{8,11}), 1.85 (m, 1 H, CH^{β}), 1.70 (m, 1 H, CH^{β}), 1.60 (m, 2 H, CH₂³), 1.55 (m, 2 H, CH₂^{δ}), 1.43 (s, 9 H, C(CH₃)₃), 1.40 (m, 2 H, CH₂^{γ}), 1.27 (m, 20 H, 10 CH₂^{Tail}), 0.87 (m, 3 H, J=6.6, CH₃¹⁸). HRMS (+ES): 533.40327 calculated for C₂₉H₅₄O₅N₂Na found 533.39110.

### Example 13

### RG 00/281

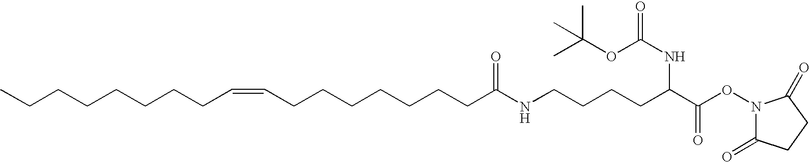

To a solution of N-α-(tert-butyloxycarbonyl)-N-ε-oleoyl-L-Lysine (1.80 g, 3.52 mmol) in THF (80 mL) were added successively N-hydroxysuccinimide (0.41 g, 3.56 mmol, 1.01 eq.) and DCC (0.73 g, 3.54 mmol, 1.01 eq.). The reaction was stirred for 16 h at RT. The precipitate was filtered and washed with EtOAc (30 mL). The filtrate was concentrated and redissolved in EtOAc and filtered again. The residue was dissolved in CHCl₃ and precipitated with Et₂O to give N-α-oleate-N-ε-(tert-butyloxycarbonyl)-L-Lysinyl succinimidate as a white solid. Yield: 1.98 g (93%). ¹H NMR (400 MHz, CDCl₃): d 5.80 (t, 1 H, J=8.0, NH^{ε}), 5.32 (m, 2 H, CH^{9,10}), 5.12 (d, 1 H, J=7.8, NH^{α}), 4.66 (m, 1 H, CH^{α}), 3.24 (q, 2 H, J=8.0, CH₂^{ε}), 2.82 (s, 4 H, 2 CH₂^{Su}), 2.14 (t, 2 H, J=6.8, CH₂²), 1.98 (m, 4 H, CH₂^{8,11}), 1.90 (m, 2 H, 2 CH^{β}), 1.60 (m, 2 H, CH₂³), 1.55 (m, 2 H, CH₂^{δ}), 1.44 (s, 9 H, C(CH₃)₃), 1.39 (m, 2 H, CH₂^{γ}), 1.25 (m, 20 H, 10 CH₂^{Tail}), 0.86 (m, 3 H, J=6.6, CH₃¹⁸).

### Example 14

### RG 00/286

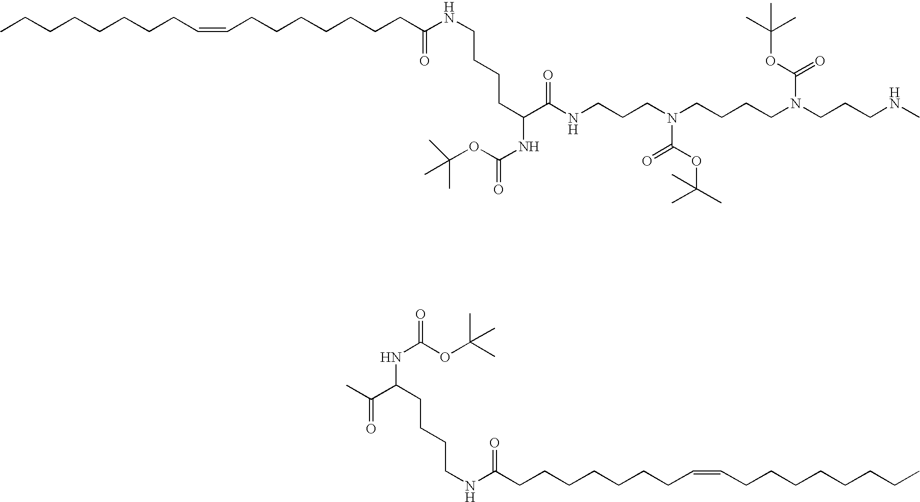

To a solution of N⁴,N⁹-bis-(tert-butyloxycarbonyl)-1,12-diamino-4,9-diazadodecane (414 mg, 0.659 mmol) in THF (60 mL) and K₂CO₃ (200 mg, 1.2 mmol, 2.2 eq.) in water (7 mL) was added a solution of N-α-(tert-butyloxycarbonyl)-N-ε-oleoyl-L-lysinyl succinimidate (800 mg, 1.32 mmol, 2.0 eq.) in THF (35 mL). The reaction was stirred overnight at RT. Most of the THF was evaporated and water (30 mL) was added. The aqueous layer was extracted with CHCl₃ (2×30 mL). The organic layer was washed with water, 0.1 M HCl, water and brine (30 mL each), dried (Na₂SO₄), filtered, evaporated and purified on SiO₂ (CHCl₃/MeOH: 95/5, Rf=0.27) to give an oil. Yield: 740 mg (0.533 mmol, 81%). ¹H NMR (400 MHz, CDCl₃): δ 7.20 (bs, 2 H, NHC^{1′}), 5.72 (bs, 2 H, NH^{ε}), 5.33 (m, 4 H, 2 CH^{9,10}), 5.25 (bs, 2 H, 2 NH^{α}), 4.08 (m, 2 H, 2 CH^{α}), 3.24 (m, 12 H, 2 CH₂^{1′}, 2 CH₂^{4′} and 2 CH₂^{ε}), 3.12 (m, 4 H, 2 CH₂^{3′}), 2.13 (t, 4 H, J=6.8, 2 CH₂²), 1.98 (m, 8 H, 2 CH₂^{8,11}), 1.80 (m, 2 H, 2 CH^{β}), 1.60 (m, 10 H, 2 CH₂^{2′}, 2 CH^{β} and 2 CH₂³), 1.50 (m, 4 H, 2 CH₂^{δ}), 1.47 (m, 4 H, 2 CH₂^{5′}), 1.45 (s, 18 H, 2 C(CH₃)₃), 1.42 (s, 18 H, 2 C(CH₃)₃), 1.37 (m, 4 H, 2 CH₂^{γ}), 1.25 (m, 40 H, 2×10 CH₂^{Tail}), 0.86 (m, 6 H, J=6.6, 2 CH₃¹⁸).

### Example 15

### RG 00/320: GSC 101

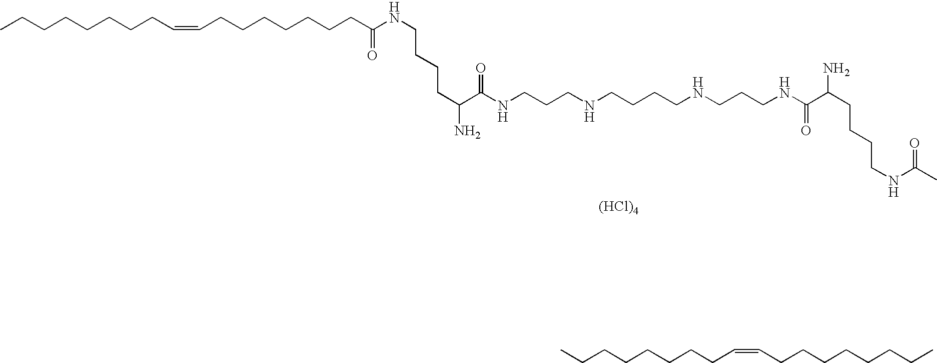

To a solution of RG 00/296 (750 mg, 0.540 mmol) in MeOH (10 mL) was added concentrated HCl (10 mL). The reaction was stirred at RT for 1 h and then evaporated. The residue was redissolved in water (60 mL) and filtered. Water was evaporated and the residue dissolved in a small amount of MeOH and precipitated with Et₂O to give a yellow solid. Yield: 533 mg (0.470 mmol, 90%). ¹H NMR (400 MHz, d₆-DMSO): δ 9.02 (m, 4 H, 2 NH₂⁺), 8.83 (t, 2 H, J=6.0, 2 NHC^{1′}), 8.30 (d, 6 H, J=4.0, 2 N^{α}H₃⁺), 8.83 (t, 2 H, J=6.0, 2 N^{ε}H), 5.30 (m, 4 H, 2 CH^{9,10}), 3.70 (q, 2 H, J=7, 2 CH^{α}), 3.22 (m, 2 H, 2 CH^{1′}), 3.13 (m, 2 H, 2 CH^{1′}), 2.97 (m, 4 H, 2 CH₂^{ε}), 2.71(m, 8 H, 2 CH₂^{3′} and 2 CH₂^{4′}), 2.10 (t, 4 H, J=7.3, 2 CH₂²), 1.95 (q, 8 H, J=6.0, 2 CH₂^{8,11}), 1.82 (h, 4 H, J=7.0, 2 CH₂^{2′}), 1.68 (m, 8 H, 2 CH₂^{β} and 2 CH₂^{5′}), 1.43 (qu, 4 H, J=6.2, 2 CH₂³), 1.35 (m, 4 H, 2 CH₂^{δ}), 1.25 (m, 44 H, 2×10 CH₂^{O1} and 2 CH₂^{γ}), 0.82 (t, 6 H, 2 CH₃¹⁸). MS (+ES): 999.8 [M+Na].

### Example 16

### RG 00/518

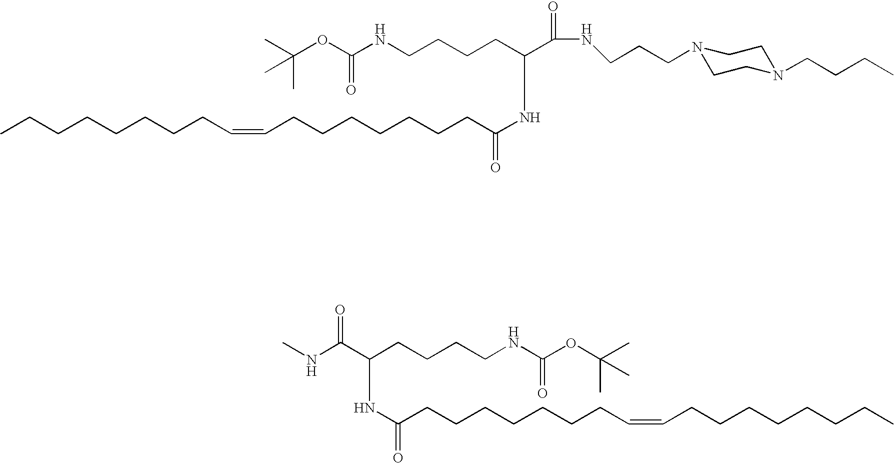

To a solution of activated aminoacid RG00/366 (610 g, 1.0 mmol) in THF (45 mL) was added bis-N-aminopropyl-piperazine (0.081 mL, 0.5 mmol, 0.5 eq.) and then potassium carbonate (0.15 g, 1.1 mmol, 2.2 eq.) in water (10 mL) and the reaction was stirred at RT for 20 h. Most of the THF was removed under vacuum, CHCl₃ was added and the organic layer was extracted, washed with water (20 mL), dried (Na₂SO₄), filtered and evaporated. The residue was purified by column chromatography on silica (CHCl₃/MeOH: 8.5/1.5, Rf=0.3) to give a white solid. Yield: 490 mg (0.413 mmol, 83%). ¹H NMR (400 MHz, CDCl₃): δ 7.68 (m, 2 H, 2 NHC¹), 6.46 (m, 2 H, 2 N^{α}H), 5.32 (m, 4 H, 2 CH^{9,10}), 4.86 (m, 2 H, 2 N^{ε}Hboc), 4.33 (q, 2 H, J=, 2 CH^{α}), 3.38 (m, 2 H, CH^{1′}), 3.28 (m, 2 H, CH^{1′}), 3.05 (m, 4 H, 2 CH₂^{ε}), 2.47 (m, 12 H, 2 CH₂^{3′} and 4 CH₂^{2′}), 2.18 (t, 4 H, J=, 2 CH₂²), 1.99 (m, 8 H) 2 CH₂^{8,10}), 1.82-1.54 (m, 12 H, 2 CH₂^{2′}, 2 CH₂³ and 2 CH₂^{β}), 1.48 (m, 4 H, 2 CH₂^{γ}), 1.42 (s, 18 H, 2 (CH₃)₃), 1.21 (m, 24 H, 10 CH₂^{O1} and 2 CH₂^{γ}), 0.87 (t, 6 H, J=6.4, 2 CH₃¹⁸). ¹³C NMR (400 MHz, CD₃OD): δ 175.2, 173.4, 157.5, 129.9, 129.8, 78.8, 56.0, 53.8, 52.9, 41.3, 40.1, 37.7, 35.9, 32.1, 31.9, 29.9, 29.6, 29.5, 29.4, 29.3, 27.9, 27.2, 26.2, 26.0, 23.3, 22.8, 13.5.

### Example 17

### RG 00/522=GSC 170

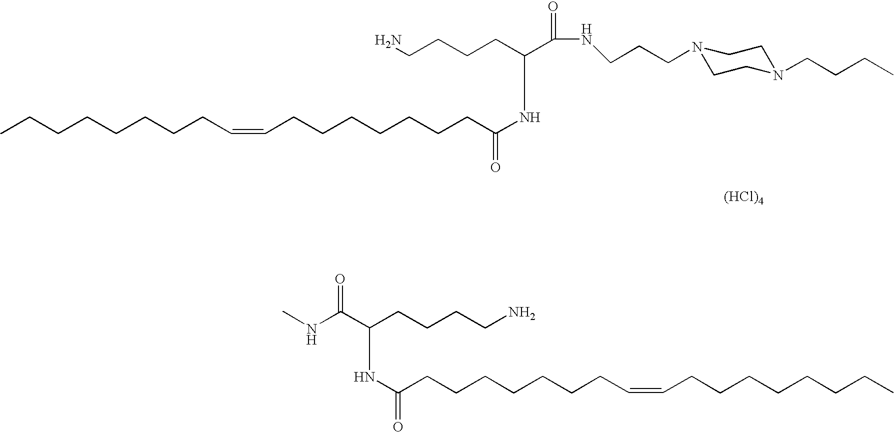

To a solution of protected RG00/518 (490 mg, 0.413 mmol) in MeOH (10 mL) was added concentrated HCl (10 mL). The reaction was stirred for 1 h and the solvent was then evaporated. The residue was redissolved in water (40 mL), filtered and evaporated. In this case it was impossible to precipitate the compound using MeOH/Et₂O. A white solid was collected. Yield: 381 mg (0.337 mmol, 81%). HRMS (+ES): 985.8879 calculated for C₅₈H₁₁₃N₈O₄, found 985.8890. Note: a similar procedure using TFA and neutralisation with K₂CO₃ was used to isolate the free amine in a quantiative yield. ¹H NMR (400 MHz, d₆-DMSO): δ 7.78 (2 d, 4 H, J=8.0, 4 NHCO), 5.29 (m, 4 H, 2 CH^{9,10}), 4.12 (q, 2 H, J=6.2, 2 CH^{α}), 3.04 (m, 4 H, 2 CH₂^{1′}), 2.47 (m, 8 H, 4 CH₂⁴), 2.29 (m, 4 H, 2 NH₂), 2.19 (t, 4 H, J=6.2, 2 CH₂^{3′}), 2.05 (m, 4 H, 2 CH₂²), 1.95 (m, 8 H, 2 CH₂^{8,10}), 1.35-1.69 (m, 12 H, 2 CH₂^{2′}, 2CH₂³ and 2 CH₂^{β}), 1.21 (m, 26 H, 10 CH₂^{O1} and CH₂^{δ} and CH₂^{γ}), 0.82 (t, 6 H, J=6.4, 2 CH₃¹⁸).

### Example 18

### RG 00/794

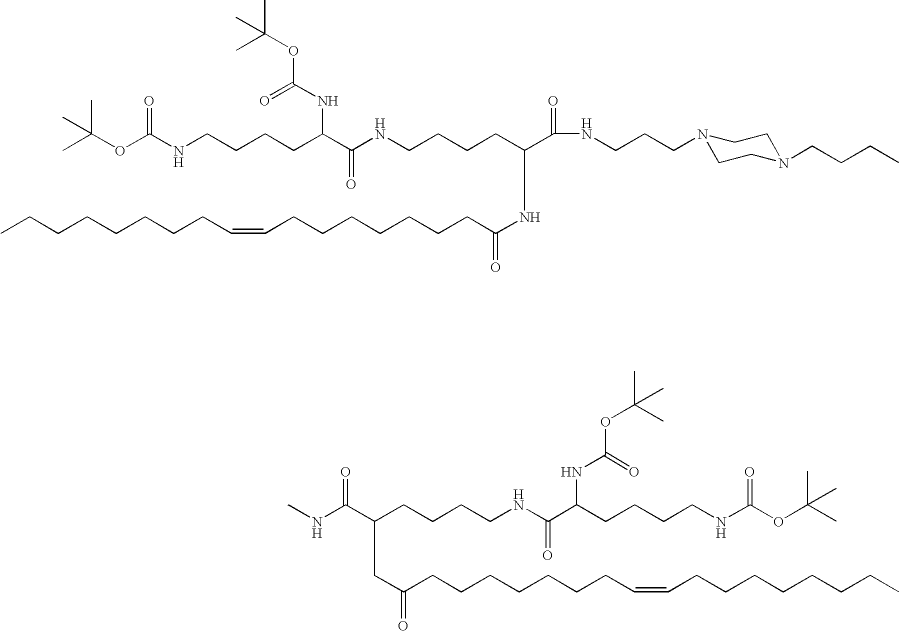

To a solution of bis aminocompound (150 mg, 0.152 mmol) in THF (40 mL) was added successively a solution of K₂CO₃ (42 mg, mmol, 2.1 eq.) in water (2 mL) and N,N-bis-(tertbutoxycarbonyl)-L-lysinyl succinimidate (140 mg, 0.304 mmol, 2.0 eq.) in THF (10 mL). The reaction was then stirred for 16 h at RT. Most of THF was evaporated and the residue redissolved in CHCl₃. Water (10 mL) was added and the organic layer extracted, washed with water (2×10 mL) and brine (20 mL). After drying (Na₂SO₄), filtration and evaporation, the residue is purified on SiO₂ (eluent: CHCl₃/MeOH/NH₄OH: 87/12/1, Rf=0.28). Et₂O is then added and the resulting white solid filtered off. Yield: 0.124 g (0.076 mmol, 50%). ¹H NMR (400 MHz, d⁶-DMSO): δ 7.75 (m, 4 H, 2 NH^{α1} and 2 NHC¹), 7.68 (t, 2 H, J=, 2 NH^{ε1}), 6.69 (t, 2 H, J=, 2 NH^{ε2} ), 6.63 (d, 2 H, J=, 2 NH^{α2}), 5.29 (m, 4 H, 2 CH^{9,10}), 4.10 (q, 2 H, J=, 2 CH^{α1}), 3.78 (q, 2 H, J=2 CH^{α2}), 3.00 (m, 6 H, 2 CH₂^{ε1} and 2 CH^{1′}), 2.95 (m, 2 H, 2 CH^{1′}), 2.84 (m, 4 H, 2 CH₂^{ε2}), 2.29 (m, 8 H, 4 CH₂^{4′}), 2.19 (m, 4 H, 2 CH₂^{3′}), 2.06 (t, 4 H, J=, 2 CH₂²), 1.95 (m, 8 H, 2 CH₂^{8,10}), 1.55-1.4 (m, 16 H), 1.32 (s, 36 H, 4 C(CH₃)₃), 1.20 (m, 48 H, 0.82 (t, 6 H, J=6.4, 2 CH₃¹⁸).

### Example 19

### RG00/813=GSC 184

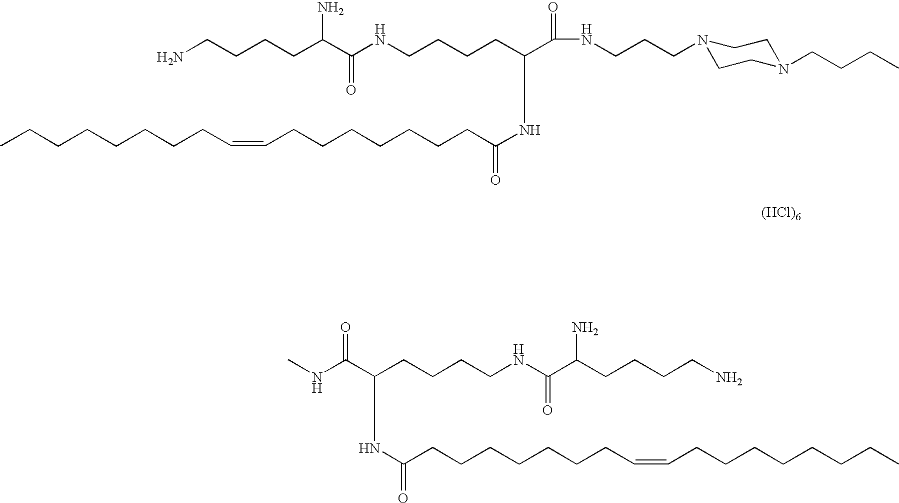

To a solution of RG00/794 (124 mg, 0.0755 mmol) in MeOH (5 mL) was added concentrated HCl (5 mL). The reaction was stirred at RT for 1 h and the solvent were then removed under vacuum. The residue was dissolved in water, filtered and evaporated. The compound was dissolved in a minimum amount of MeOH and precipitated with Et₂O. The resulting solid was filtered and collected. Yield: 0.102 g (0.070 mmol, 93%). ¹H NMR (400 MHz, d₆-DMSO): δ 8.66 (d, 2 H, J=7.8, 2 NH^{ε1}), 8.28 (m, 6 H, 2 N^{α}H₃⁺), 8.09 (m, 2 H, 2 NHC¹), 8.05 (m, 6 H, 2 N^{ε}H₃⁺), 7.98 (d, 2 H, J=7.0, 2 N^{α}H), 5.29 (m, 4 H, 2 CH^{9,10}), 4.09 (m, 2 H, 2 CH^{α1}), 3.72 (m, 2 H, 2 CH^{α2}), 3.65 (m, 2 H, 2 NH⁺), 3.10 (m, 12 H, 2 CH₂^{ε1}, 2 CH₂^{3′} and 2 CH₂^{1′}), 2.74 (m, 8 H, 2 CH₂^{ε2}), 2.11 (t, 4 H, J=7.2, 2 CH₂²), 1.95 (m, 8 H, 2 CH₂^{8,10}), 1.82 (m, 2 H, 2 CH₂^{δ1}), 1.70 (m, 2 H, 2 CH₂^{β2}), 1.57 (m, 6 H, 2 CH₂^{δ2} and 2 CH^{β1}), 1.50-1.15 (m, 66 H), 0.84 (t, 6 H, J=6.4, 2 CH₃¹⁸). MS (+ES): 1264.9 [M+Na].

### Example 20

### RG 00/787

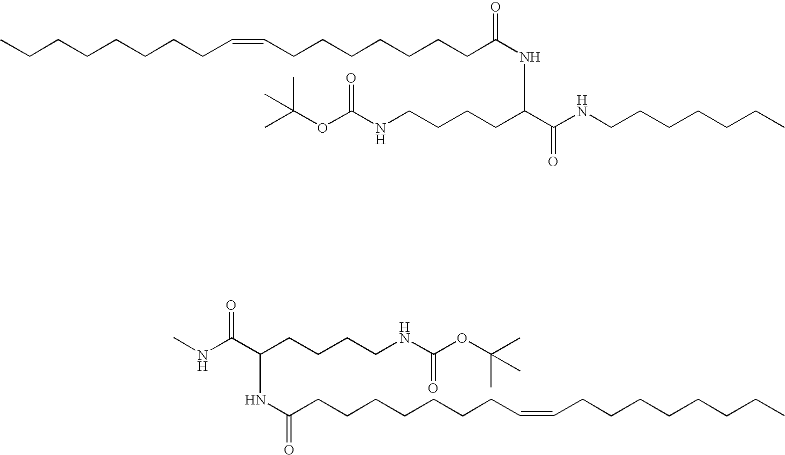

To a solution of 1,6-diaminohexane (72 mg, 0.62 mmol) in THF (60 mL) and K₂CO₃ (180 mg, 1.30 mmol, 2.1 eq.) in water (10 mL) was added a solution of N-α-oleoyl-N-ε-(tert-butyloxycarbonyl)-L-lysinyl succinimidate (750 mg, 1.23 mmol, 2 eq.). The reaction was stirred overnight at RT. Most of the THF was evaporated and water (30 mL) was added. The aqueous layer was extracted with CHCl₃ (2×50 mL). The organic layer was washed with water, 0.1 M HCl, water and brine (20 mL each), dried (Na₂SO₄), filtered, evaporated and purified by column chromatography on SiO₂ (CHCl₃/MeOH: 9/1, Rf=0.33) to give an oil. Yield: 650 mg (0.59 mmol, 95%). ¹H NMR (400 MHz, d₆-DMSO): δ 7.73 (m, 4 H, 2 N^{α}H and 2 N^{1′}H), 6.68 (t, 2 H, J=5.0, 2 N^{ε}H), 5.28 (m, 4 H, 2 CH^{9,10}), 4.12 (m, 2 H, 2 CH^{α}), 2.99 (q, 4 H, J=6.4, 2 CH^{1′}), 2.83 (q, 4 H, J=6.6, 2 CH₂^{ε}), 2.07 (dt, 4 H, J=3.2, 7.0, 2 CH₂²), 1.95 (m, 8 H, 2 CH₂^{8,11}), 1.52 (m, 2 H, 2 CH^{β}), 1.42 (m, 6 H, 2 CH₂³ and 2 CH^{β}), 1.32 (s, 18 H, 2 C(CH₃)₃), 1.31-1.15 (m, 56 H, 2×10 CH₂^{Tail}, 2 CH₂^{δ}, 2 CH₂^{γ}, 2 CH₂^{2′} and 2 CH₂^{3′}), 0.82 (t, 6 H, J=6.8, 2 CH₃¹⁸).

### Example 21

### RG 00/873: GSN 14

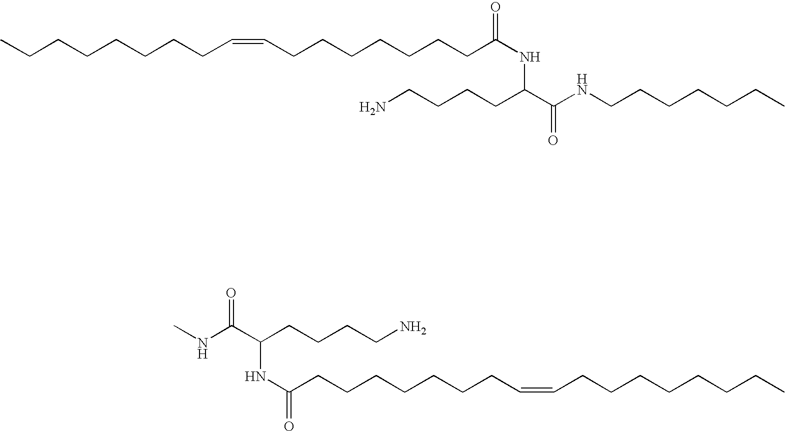

To a solution of protected compound (640 mg, 0.581 mmol) in CH₂Cl₂ (10 mL) was added TFA (10 mL). The reaction was stirred at RT for 1 h and then evaporated (using several Et₂O (10 mL) to coevaporate). The oily residue was then dissolved in CH₂Cl₂, washed with 10% aqueous K₂CO₃ (10 mL), water and brine. The organic phase was dried (Na₂SO₄), filtered and evaporated to give a pale brown solid which was triturated with Et₂O, filtered and dried to give a white solid. Yield: 460 mg (0.510 mmol, 88%). The deprotection can be carried out using concentrated HCl in methanol giving the hydrochloric salt named GSN 14. ¹H NMR (400 MHz, d₆-DMSO): δ 7.80 (m, 4 H, 2 N^{α}H and 2 N^{1′}H), 5.28 (m, 4 H, 2 CH^{9,10}), 4.16 (m, 2 H, 2 CH^{α}), 3.20 (bs, 4 H, 2 NH₂), 2.99 (q, 4 H, J=6.4, 2 CH^{1′}), 2.53 (m, 4 H, 2 CH₂^{ε}), 2.10 (dt, 4 H, J=3.2, 7.0, 2 CH₂²), 1.91 (m, 8 H, 2 CH₂^{8,11}), 1.52 (m, 2 H, 2 CH^{β}), 1.48 (m, 2 H, 2 CH^{β}), 1.42 (m, 4 H, 2 CH₂³), 1.31-1.15 (m, 56 H, 2×10 CH₂^{Tail}, 2 CH₂^{δ}, 2 CH₂^{γ}, 2 CH₂^{2′} and 2 CH₂^{3′}), 0.81 (t, 6 H, J=6.8, 2 CH₃¹⁸).

### Example 22

### RG 00/874

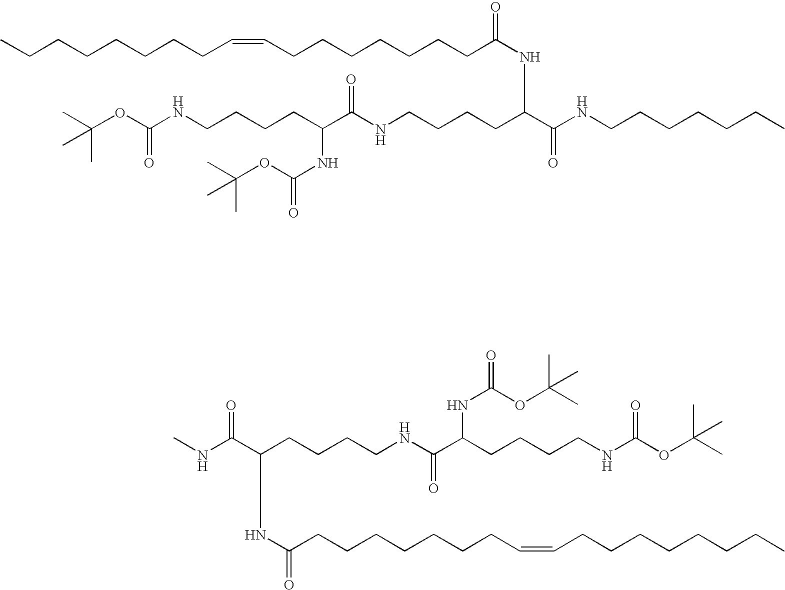

To a 1/9 mixture of water and THF (20 mL) containing RG 00/873 (100 mg, 0.111 mmol) and potassium carbonate (32 mg, 0.232 mmol, 2.1 eq.) was added N,N-bis-tert-butyloxycarbonyl)-L-lysinyl succinimidate (103 mg, 0.232 mmol, 2.1 eq.). The reaction was stirred for 20 h at RT. Most of THF was removed and the residue diluted with water (10 mL) and CHCl₃ (40 mL). The organic layer was decanted and washed successively with water (10 mL), 0.1 M HCl (20 mL), water (10 mL) and brine (25 mL). The organic layer was dried over sodium sulphate, filtered and evaporated. The resulting oil was crystallised from Et₂O. The white solid was collected. Yield: 164 mg (0.105 mmol, 95%).

### Example 23

### RG 00/875=GSC 197

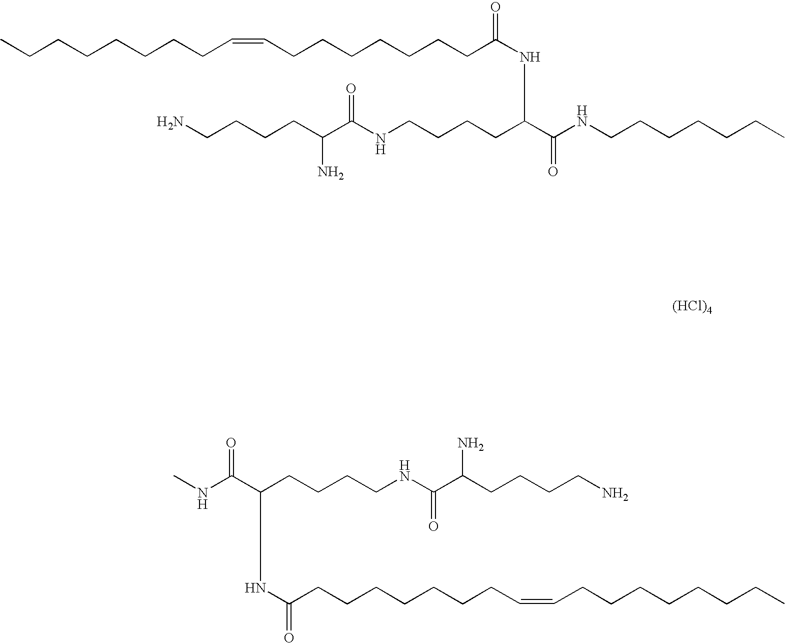

To a solution of RG 00/874 (160 mg, 0.103 mmol) in methanol (5 mL) is added concentrated HCl (5 mL). The reaction is stirred for 1 h and then evaporated to dryness. The residue is then dissolved in water (30 mL), filtered on sintered frit funnel (N^{o} 3), evaporated to dryness using EtOH to coevaporate. The residue is dissolved in a small amount of methanol and precipitated with Et₂O to give the desired compound as a pale brown solid. Yield: 124 mg (0.951 mmol, 95%). ¹H NMR (400 MHz, d₆-DMSO): δ 8.59 (t, 2 H, J=5.0, 2 N^{1′}H), 8.22 (m, 6 H, 2 N^{α2}H₃⁺), 7.96 (m, 6 H, 2 N^{ε2}H₃⁺), 7.89 (d, 2 H, J=8.0, 2 N^{α1}H), 7.89 (t, 2 H, J=5.8, 2 N^{ε1}H), 5.29 (m, 4 H, 2 CH^{9,10}), 4.14 (dt, 2 H, J=5.4, 8.0, 2 CH^{α1}), 3.70 (m, 2 H, 2 CH^{α2}), 3.05 (m, 4 H, 2 CH₂^{1′}), 2.98 (q, 4 H, J=5.8, 2 CH₂^{ε1}), 2.72 (m, 4 H, 2 CH₂^{ε2}), 2.09 (t, 4 H, J=7.0, 2 CH₂²), 1.94 (m, 8 H, 2 CH₂^{8,11}), 1.69 (m, 2 H, 2 CH₂^{β2}), 1.55 (m, 6 H, 2 CH₂^{δ2} and 2 CH^{β1}), 1.48-1.15 (m, 56 H), 0.81 (t, 6 H, J=6.6, 2 CH₃¹⁸).

### Example 24

### RG 00/804

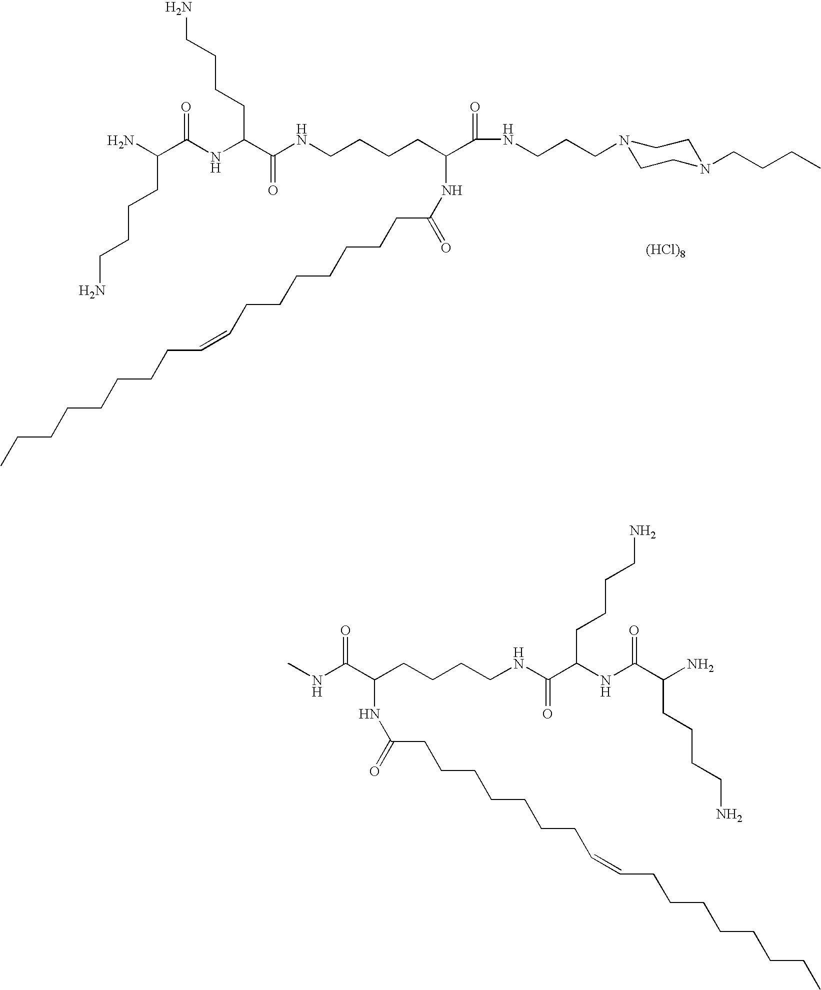

To a solution of RG00/794 (110 mg, 0.052 mmol) in MeOH (7 mL) was added concentrated HCl (7 mL). The reaction was stirred at RT for 1 h and the solvent were then removed under vaccuum using EtOH to coevaporate. The residue was dissolved in water, filtered and evaporated. The compound was dissolved in a minimum amount of MeOH and precipitated with Et₂O. The resulting solid was filtered and collected as a white powder. Yield: 88 mg (0.049 mmol, 94%). ¹H NMR (400 MHz, d₆-DMSO): δ 8.69 (d, 2 H, J=7.8, 2 NH^{α2}), 8.30 (m, 2 H, 2 NH^{α1}), 8.12 (m, 2 H, 2 NHC¹), 7.95 (m, 12 H, 2 NH^{ε3} , 2 NH^{ε3} and NH^{α3}), 5.29 (m, 4 H, 2 CH^{9,10}), 4.20 (m, 2 H, 2 CH^{α1}), 4.08 (m, 2 H, 2 CH^{α3}), 3.84 (m, 2 H, 2 CH^{α2}), 3.65 (m, 2 H, 2 NH⁺), 3.10 (m, 12 H, 2 CH₂^{3′} and 2 CH₂^{4′}), 3.05 (m, 2 H, 2 CH^{1′}), 2.95 (m, 2 H, 2 CH^{1′}), 2.74 (m, 8 H, 2 CH₂^{ε1} and 2 CH₂^{ε2}), 2.11 (t, 4 H, J=7.2, 2 CH₂²), 1.95 (m, 8 H, 2 CH₂^{8,10}), 1.80-1.12 (m, 82 H), 0.84 (t, 6 H, J=6.4, 2 CH₃¹⁸). MS (+ES): m/z [M+H]²⁺ 750.1.

### Example 25

### RG 00/797

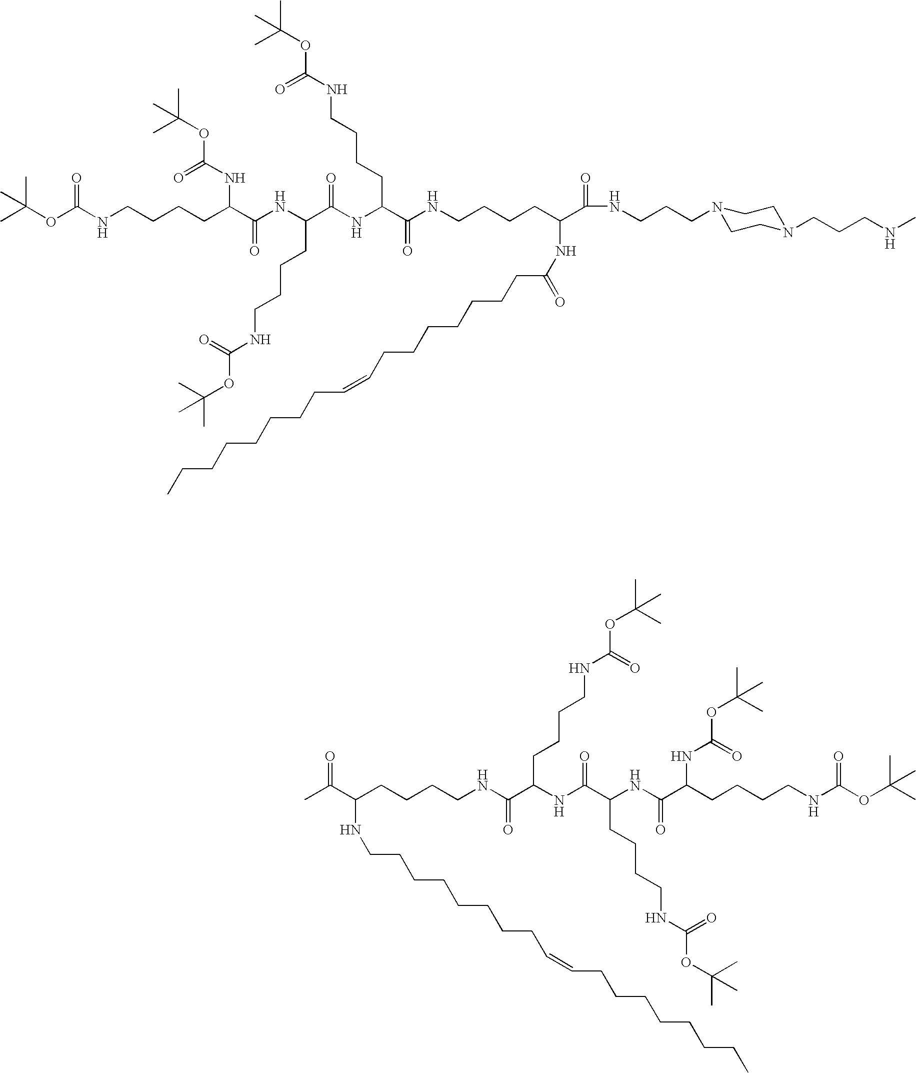

To a solution of bis aminocompound (140 mg, 0.142 mmol) in THF (48 mL) was added successively a solution of K₂CO₃ (40 mg, mmol, 2.1 eq.) in water (10 mL) and (Boc)₄-KKKOSu (256 mg, 0.284 mmol, 2.0 eq.) in THF (10 mL). The reaction was then stirred for 16 h at RT. Most of THF was evaporated and the residue redissolved in CHCl₃. Water (10 mL) was added and the organic layer was extracted, washed with 5% K₂CO₃, water (10 mL) and brine (20 mL). After drying (Na₂SO₄), filtration and evaporation, the residue was purified on SiO₂ (eluent: CHCl₃/MeOH/NEt₃: 85/15/1, Rf=0.32). Et₂O is then added and the resulting white solid filtered off. Yield: 0.310 g (0.121 mmol, 85%). ¹H NMR (400 MHz, d₆-DMSO): δ 8.00 (m, 2 H, 2 NH^{α}), 7.85 (m, 2 H, 2 NH^{α}), 7.75 (m, 4 H, 2 NH^{α} and 2 NHC¹), 6.85 (m, 2 H, 2 N^{α}H), 6.68 (m, 6 H, 2×3 N^{ε}H), 5.29 (m, 4 H, 2 CH^{9,10}), 4.18 (m, 2 H, 2 CH^{α}), 4.09 (m, 4 H, 2×2 CH^{α}), 3.82 (m, 4 H, 2×2 CH^{α}), 3.00 (m, 6 H, 2 CH₂^{ε} and 2 CH^{1′}), 2.75 (m, 2 H, 2 CH^{1′}), 2.84 (m, 12 H, 2×3 CH₂^{ε}), 2.47 (m, 8 H, 2×2 CH₂^{4′}), 2.29 (m, 4 H, 2 CH₂^{3′}), 2.09 (t, 4 H, J=9.0, 2 CH₂²), 1.95 (m, 8 H, 2 CH₂^{8,10}), 1.65-1.15 (m, 168 H), 0.82 (t, 6 H, J=6.4, 2 CH₃¹⁸).

### Example 26

### RG 00/805

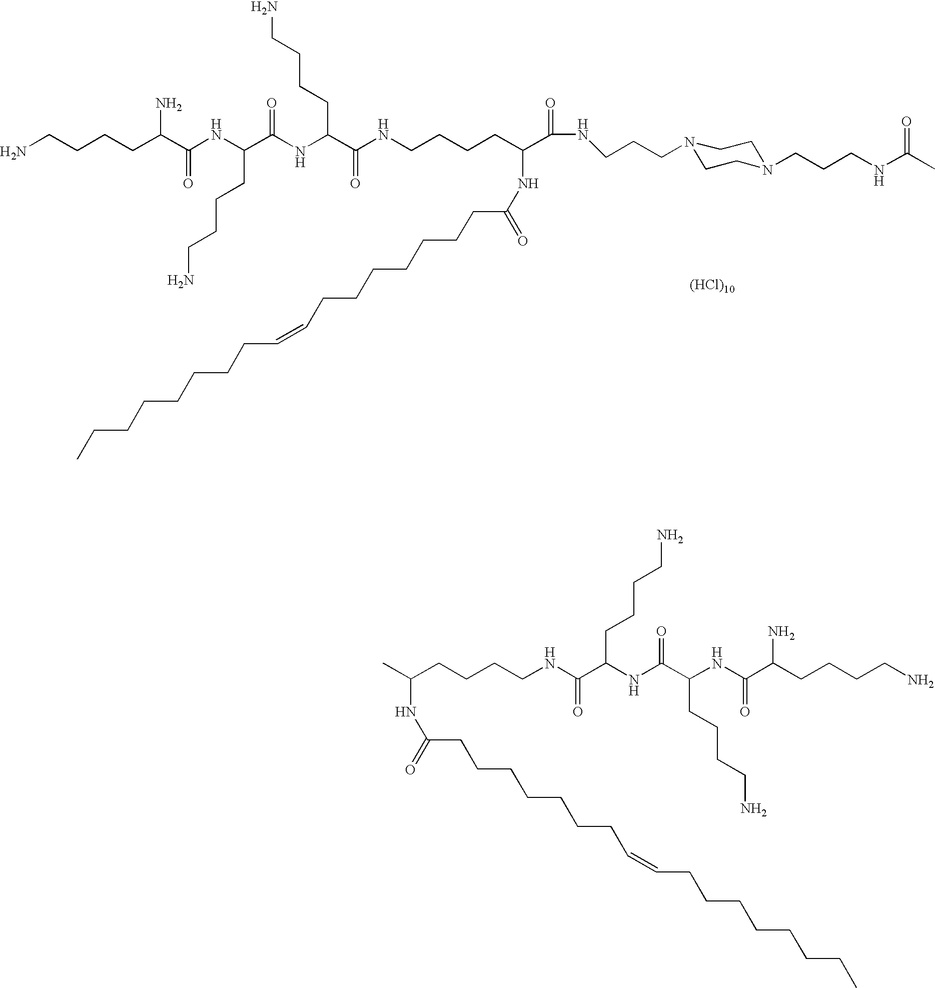

To a solution of RG00/797 (110 mg, 0.052 mmol) in MeOH (7 mL) was added concentrated HCl (7 mL). The reaction was stirred at RT for 1 h and the solvent were then removed under vaccuum using EtOH to coevaporate. The residue was dissolved in water (40 mL), filtered and evaporated. The compound was dissolved in a minimum amount of MeOH and precipitated with Et₂O. The resulting solid was filtered and collected as a pale brown powder. Yield: 88 mg (0.049 mmol, 94%). ¹H NMR (400 MHz d₆-DMSO): δ 8.80 (d, 2 H, J=7.8, 2 NH^{α}), 8.30 (m, 6 H, 2×3 NH^{α}), 8.03 (m, 14 H, 2 NHC¹ and 2×3 N^{ε}H₃⁺), 5.30 (m, 4 H, 2 CH^{9,10}), 4.28 (m, 2 H, 2 CH^{α}), 4.18 (m, 2 H, 2 CH^{α}), 4.08 (m, 2 H, 2 CH^{α}), 3.85 (m, 2 H, 2 CH^{α}), 3.65 (m, 2 H, 2 NH⁺), 3.10 (m, 16 H, 2 CH₂^{ε1}, 2 CH₂^{3′} and 2×2 CH₂^{4′}), 3.02 (m, 2 H, 2 CH^{1′}), 2.95 (m, 2 H, 2 CH^{1′}), 2.74 (m, 8 H, 2×3 CH₂^{ε}), 2.10 (t, 4 H, J=7.2, 2 CH₂²), 1.95 (m, 8 H, 2 CH₂^{8,10}), 1.71 (m, 4 H, 2 CH₂^{β}), 1.60-1.17 (m, 108 H), 0.84 (t, 6 H, J=6.4, 2 CH₃¹⁸). MS (+ES): m/z [M+H]²⁺ 750.1.

### Example 27

### RG 00/823

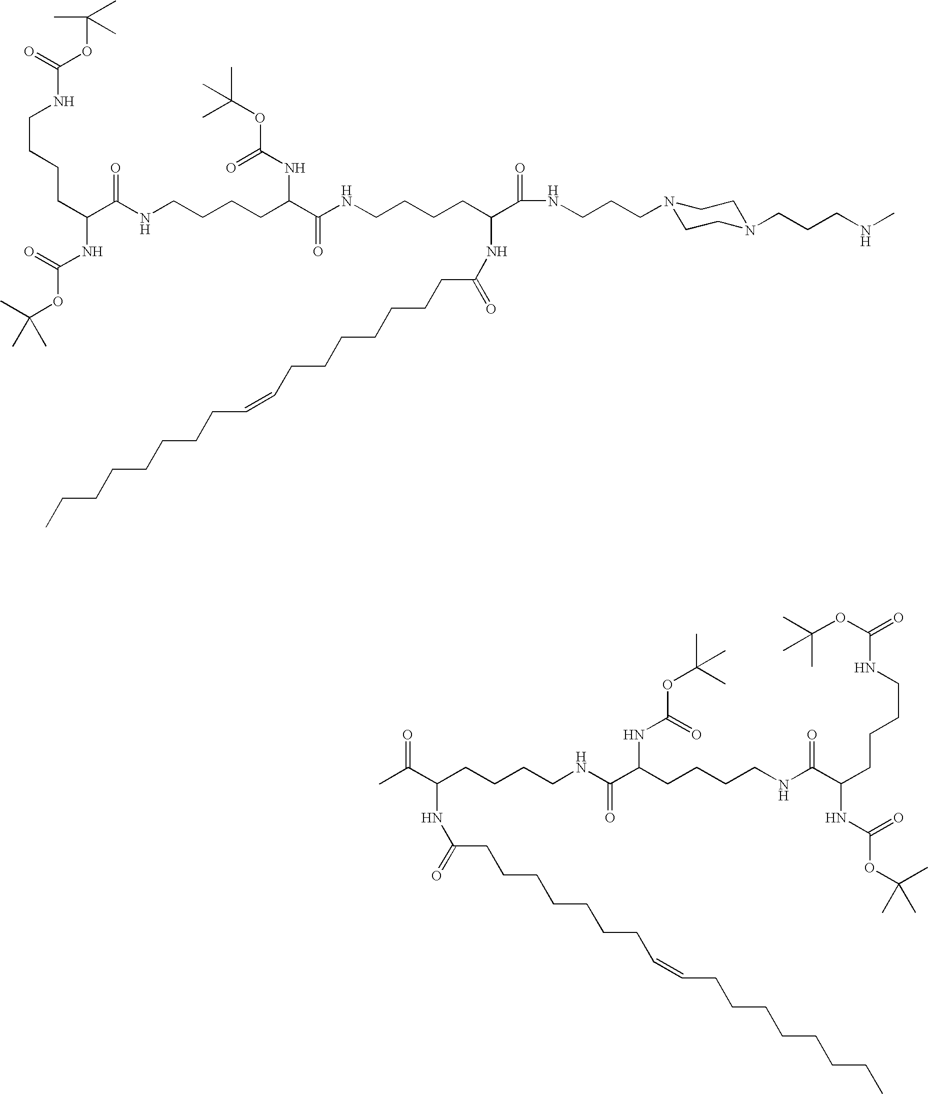

To a solution of bis aminocompound (76 mg, 0.077 mmol) in THF (40 mL) was added successively a solution of K₂CO₃ (22 mg, 0.159 mmol, 2.06 eq.) in water (2 mL) and Boc₃(K-ε-K)-OSu (105 mg, 0.156 mmol, 2.0 eq.) in THF (8 mL). The reaction was then stirred for 16 h at RT. Most of THF was evaporated and the residue redissolved in CHCl₃. Water (10 mL) was added and the organic layer extracted, washed with water (2×10 mL) and brine (20 mL). After drying (Na₂SO₄), filtration and evaporation, the residue was purified on SiO₂ (eluent: CHCl₃/MeOH/NEt₃: 91/8/1, Rf=0.30). Et₂O is then added and the resulting white solid filtered off. Yield: 0.124 g (0.059 mmol, 77%). ¹H NMR (400 MHz, d₆-DMSO): δ 7.79 (m, 4 H, 2 NH^{α} and 2 NH^{ε}), 7.67 (m, 4 H 2 NHC¹ and 2 NH^{ε}), 6.69 (m, 2 H, 2 NH^{ε}), 8.28 (m, 8 H, 2×2 NH^{α}), 5.28 (m, 4 H, 2 CH^{9,10}), 4.10 (m, 2 H, 2 CH^{α1}), 3.78 (m, 4 H, 2×2 CH^{α}), 3.00 (m, 6 H, 2 CH^{ε} and 2 CH^{1′}), 2.98 (m, 2 H, 2 CH^{1′}), 3.13 (m, 12 H, 2×2 CH₂^{ε}), 2.47 (m, 4 H, 2 CH₂^{3′}), 2.25 (m, 8 H, 2×2 CH₂^{4′}), 2.08 (t, 4 H, J=7.2, 2 CH₂²), 1.95 (m, 8 H, 2 CH₂^{8,10}), 1.80-1.14 (m, 138 H), 0.82 (t, 6 H, J=6.4, 2 CH₃¹⁸).

### Example 28

### RG 00/830

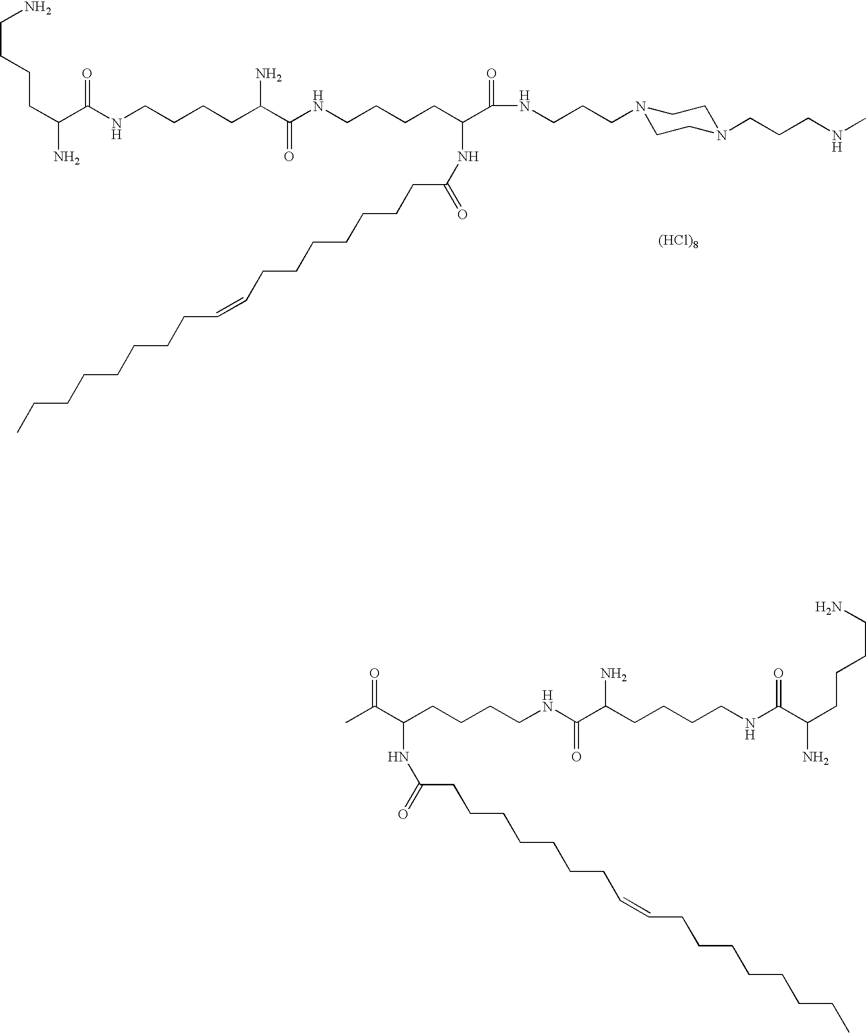

To a solution of RG00/823 (124 mg, 0.059 mmol) in MeOH (10 mL) was added concentrated HCl (6 mL). The reaction was stirred at RT for 1 h and the solvent were then removed under vaccuum using EtOH to coevaporate. The residue was dissolved in water (40 mL), filtered and evaporated. The compound was dissolved in a minimum amount of MeOH and precipitated with Et₂O. The resulting solid was filtered and collected as a pale pink powder. Yield: 101 mg (0.056 mmol, 96%). ¹H NMR (400 MHz, d₆-DMSO): δ 8.73 (t, 2 H, J=7.8, 2 NH^{ε}), 8.67 (m, 2 H 2 NH^{ε1}), 8.28 (m, 8 H, 4 NH₂^{α}), 8.15 (m, 6 H, 2 NHC¹, 2 NH^{ε2} and 2 NH^{ε3}), 7.99 (m, 2 H, 2 NH^{α1}), 5.29 (m, 4 H, 2 CH^{9,10}), 4.09 (m, 2 H, 2 CH^{α1}), 3.72 (m, 4 H, 2 CH^{α3} and 2 CH^{α2}), 3.65 (m, 2H, 2 NH⁺), 3.08 (m, 10 H, 2 CH₂^{ε1}, 2 CH₂^{ε2}, 2 CH₂^{3′} and 2 CH₂^{4′}), 2.74 (m, 2 H, 2 CH₂^{1′}), 2.11 (t, 4 H, J=7.2, 2CH₂²), 1.95 (m, 8 H 2 CH₂^{8,10}), 1.80-1.14 (m, 82 H), 0.83 (t, 6 H, J=6.4, 2 CH₃¹⁸).

Examples 29 to 40 describe alternative routes for the synthesis of GSC170 and derivatives thereof.

### Example 29 (RG00/781)

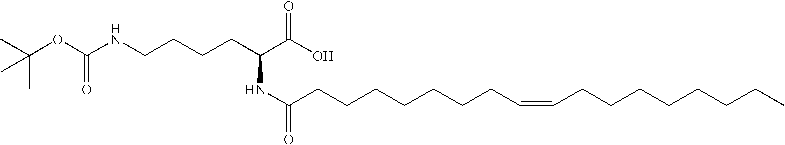

To a solution of N-ε-(t-butoxycarbonyl)-L-lysine (446 mg, 1.89 mmol) in THF (53 mL) were added successively a solution of K₂CO₃ (288 mg, 2.08 mmol) in water (8 mL) and oleoyl succinimidate (754 mg, 1.99 mmol). The reaction was stirred at RT for 15 h and the most of THF was evaporated. Water and CHCl₃ (25 mL each) were added and the mixture was acidified with 1 M HCl to pH: 2. The organic layer was separated, and the aqueous layer was extracted with CHCl₃ (2×20 ml). The organic layer was washed with brine (15 mL), dried (Na₂SO₄), filtrated and evaporated to give a white solid. Yield: 940 mg (1.84 mmol, 97%). Rf(SiO₂): 0.28 (CHCl₃—MeOH 92:8).

RMN-¹H (500 MHz, CDCl₃), δ (ppm): 5.33 (m, 2 H, CH^{9,10}), 4.15 (m, 1 H, CH^{α}), 3.05 (m, 2 H, CH₂^{ε}), 2.15 (m, 2 H, J=6.0 Hz, CH₂²), 1.98 (m, 4 H, CH₂^{8,11}), 1.80-1.48 (m, 4 H, CH₂³, CH₂^{β}), 1.46-1.20 (m, 33 H, CH₂^{γ}, CH₂^{δ}, C(CH₃)₃, 10 CH₂^{ol}), 0.84 (t, 3 H, J=6.8 Hz, CH₃¹⁸). RMN-¹³C (125 MHz, CDCl₃), δ (ppm): 178.6, 174.4, 156.2, 129.9, 129.7, 78.9, 54.5, 40.0, 36.4, 31.9, 31.3, 29.8, 29.5, 29.4, 29.3, 28.5, 27.3, 27.2, 25.9, 25.0, 22.9, 22.7, 14.1. HRMS (+ESI): C₂₉H₅₄N₂O₅Na [M+Na], calcd.: m/z=533.3930, found: m/z=533.3903

### Example 30 (RG00/518)

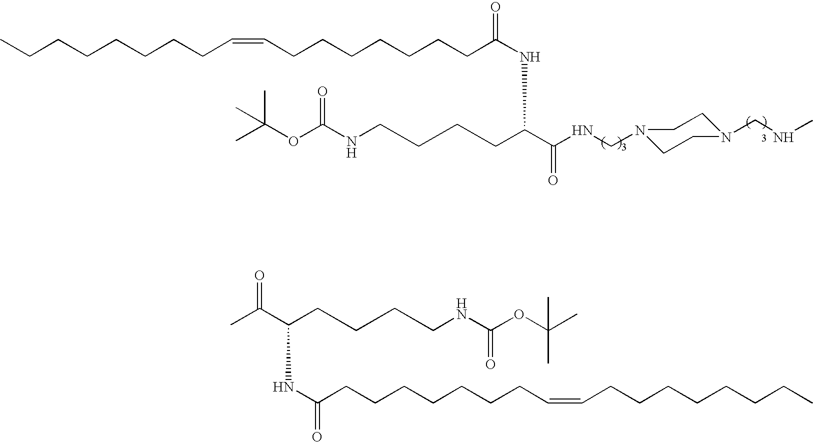

To a solution of aminoacid RG00/781 (410 mg, 0.80 mmol) in CH₂Cl₂ (4 ml) were added benzotriazole-1-yl-oxi-tris-pyrrolidino-phosphonium hexafluorphosphate (418 mg, 0.80 mmol), DIEA (280 μl, 1.61 mmol) and 1,4-Bis(3-aminopropyl)piperazine (64 μl, 0.31 mmol). The mixture was stirred at RT for 16 h. The solvent was removed, CHCl₃ (40 mL) was added and the organic layer was washed with 5% NaHCO₃ (3×12 mL) and brine (30 mL), dried (Na₂SO₄), filtrated and evaporated. The residue was purified by column chromatography on reverse phase (C-18) to give a syrup. Yield: 295 mg (0.25 mmol, 81%), Rf (SiO₂): 0.46 (CHCl₃—MeOH 85:15), Rf (C-18): 0.15 (MeOH). RMN-¹H (500 MHz, CDCl₃), δ (ppm): 7.49 (m, 2 H, NHCO), 6.52 (m, 2 H, N^{α}HCO), 5.32 (m, 4 H, 2 CH^{9,10}), 4.81 (m, 2 H, NHBOC), 4.29 (m, 2 H, CH^{α}), 3.30 (m, 4 H, 2 CH₂^{1′}), 3.05 (m, 4 H, J=6.0 Hz, 2 CH₂^{ε}), 2.76 (s broad, 8 H, 4 CH₂^{4′}), 2.61 (s broad, H, 2 CH₂^{3′}), 2.19 (t, 4 H, J=7.4 Hz, 2 CH₂²), 1.97 (m, 8 H, 2 CH₂^{8,11}), 1.72 (m, 6 H, 2 CH₂^{2′}, 2 CHₐ^{β}), 1.68 (m, 6 H, 2 CH₂³, 2 CH_{b}^{β}), 1.52-1.15 (m, 66 H, 2 CH₂^{γ}, 2 CH₂^{δ}, 2×10 CH₂^{o1}, 2 C(CH₃)₃), 0.85 (t, 6 H, J=6.7 Hz, 2 CH₃¹⁸). RMN-¹³C (125 MHz, CDCl₃), δ (ppm): 173.6, 172.3, 156.2, 130.0, 129.7, 79.0, 55.7, 53.1, 52.0, 40.0, 38.0, 36.5, 32.0, 31.9, 29.7, 29.5, 29.3, 29.2, 28.4, 27.2, 25.8, 25.7, 24.8, 22.7, 22.6, 14.1. HRMS (+ESI): C₆₈H₁₂₉N₈O₈ [M+H], calcd.: m/z=1185.9928, found: m/z=1186.0006.

### Example 31 (GSC170)

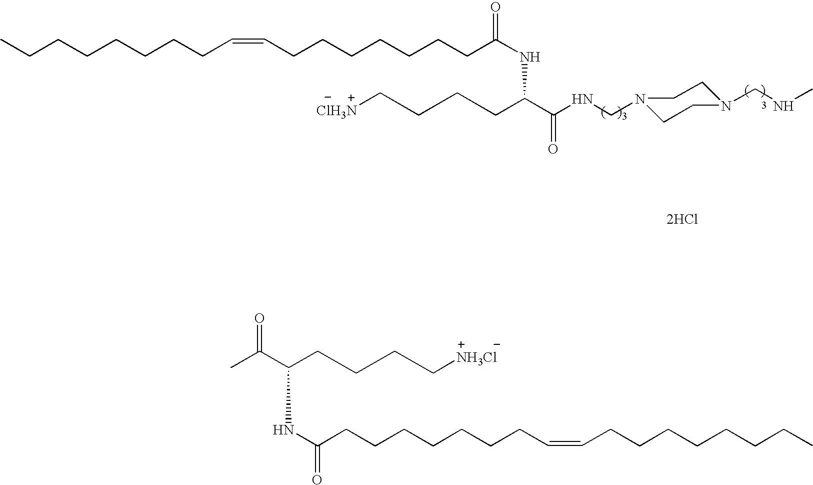

To a solution of RG00/518 (710 mg, 0.60 mmol) in EtOAc (50 mL) was added 4.9 N HCl-EtOAc (50 ml). The reaction was stirred at RT for 2 h. The precipitate was filtrated and washed with ether to give a white solid. Yield 542 mg (0.48 mmol, 80%), Rf (SiO₂): 0.63 (MeOH—NH₄OH 80:20).

RMN-¹H (500 MHz, DMSO-d₆), δ (ppm): 8.12 (m, 2 H, NHCO), 8.05-7.87 (m, 8 H, N^{α}HCO, 2 N^{ε}H₃⁺), 5.31 (m, 4 H, 2 CH^{9,10}), 4.10 (m, 2 H, J=5.0 Hz, 8.4 Hz, 2 CH^{α}), 3.70 (s broad, 4 H, 4 CHₐₓᵢₐₗ^{4′}), 3.44 (s broad, 4 H, 4 CH_{ecuat.}^{4′}), 3.10 (m, 8 H, 2 CH₂^{1′}, 2 CH₂^{3′}), 2.72 (m, 4 H, J=6.2 Hz, 2 CH₂^{ε}), 2.12 (m, 4 H, J=6.8 Hz, 2 CH₂²), 1.95 (m, 8 H, 2 CH₂^{8,11}), 1.84 (m, 4 H, J=6.3 Hz, 2 CH₂^{2′}), 1.65-1.40 (m, 12 H, 2 CH₂^{β}, 2 CH₂^{δ}, 2 CH₂³), 1.35-1.15 (m, 44 H, 2 CH₂^{γ}, 2×10 CH₂^{o1}), 0.83 (t, 6 H, J=6.7 Hz, 2 CH₃¹⁸).

RMN-¹³C (125 MHz DMSO-d₆), δ (ppm): 172.5, 172.1, 129.7, 55.0, 53.7, 52.7, 48.1, 40.1, 39.9, 39.8, 39.6, 39.4, 39.3, 39.1, 38.5, 31.4, 29.3, 29.2, 28.9, 28.8, 28.7, 28.7, 26.7, 25.3, 22.2, 14.1. HRMS (+ESI): C₅₈H₁₁₃N₈O₄ [M+H], calcd.: m/z=985.8879, found: m/z=985.8805.

### Example 32 (Compound 4)

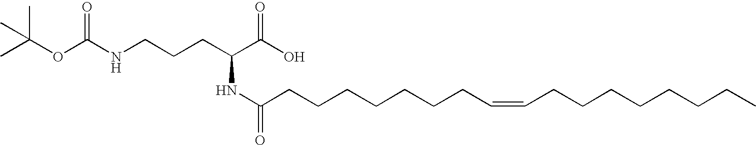

To a solution of N-δ-(t-butoxycarbonyl)-L-ornitine (440 mg, 1.89 mmol) in THF (53 mL) were added successively a solution of K₂CO₃ (288 mg, 2.08 mmol) in water (8 mL) and oleoyl succinimidate (755 mg, 1.99 mmol). The reaction was stirred at RT for 15 h and the most of THF was evaporated. Water and CHCl₃ (25 mL each) were added and the mixture was acidified with 1 M HCl to pH: 2. The organic layer was separated, and the aqueous layer was extracted with CHCl₃ (2×20 ml). The organic layer was washed with brine (15 mL), dried (Na₂SO₄), filtrated and evaporated to give a white solid. Yield: 898 mg (1.81 mmol, 96%). Rf(SiO₂): 0.16 (CHCl₃—MeOH 92:8).

RMN-¹H (500 MHz, CDCl₃), δ (ppm): 7.03 (m, 1 H, NHCO), 5.32 (m, 2 H, CH^{9,10}), 4.40 (m, 1 H, CH^{α}), 3.11 (m, 2 H, CH₂^{δ}), 2.19 (t, 2 H, J=7.4 Hz, CH₂²), 1.99 (m, 4 H, CH₂^{8,11}), 1.85 (m, 1 H CHₐ^{β}), 1.70-1.20 (m, 34 H, CH_{b}^{β}, CH₂³, CH₂^{γ}, C(CH₃)₃, 10 CH₂^{o1}), 0.87 (t, 6 H, J=6.7 Hz, CH₃¹⁸). RMN-¹³C (125 MHz, CDCl₃), δ (ppm): 176.6, 174.4, 156.5, 130.0, 129.7, 79.4, 53.0, 39.8, 36.4, 33.9, 31.9, 29.8, 29.5, 29.4, 29.3, 29.1, 28.9, 28.4, 27.2, 26.4, 25.7, 25.6, 24.8, 22.7, 14.1. HRMS (+ESI): C₂₈H₅₂N₂O₂ [M+H], calcd.: m/z=519.3774, found: m/z=519.3767.

### Example 33 (Compound 5)

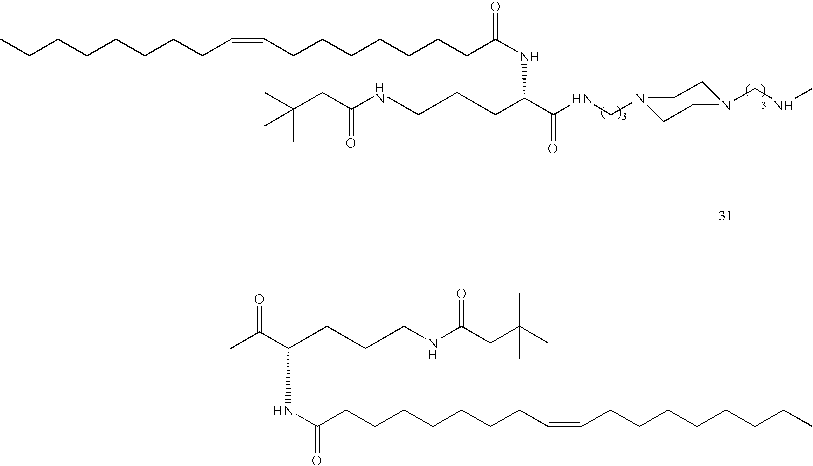

To a solution of aminoacid 4 (548 mg, 1.10 mmol) in CH₂Cl₂ (5.5 ml) were added benzotriazole-1-yl-oxi-tris-pyrrolidino-phosphonium hexafluorphosphate (574 mg, 1.10 mmol), DIEA (385 μl, 2.20 mmol) and 1,4-Bis(3-aminopropyl)piperazine (84 μl, 0.41 mmol). The mixture was stirred at RT for 18 h. The solvent was removed, CHCl₃ (40 mL) was added and the organic layer was washed with 5% NaHCO₃ (3×10 mL) and brine (25 mL), dried (Na₂SO₄), filtrated and evaporated. The residue was purified by column chromatography on reverse phase (C-18) to give a syrup. Yield: 360 mg (0.31 mmol, 76%), Rf (SiO₂): 0.46 (CHCl₃—MeOH 85:15), Rf (C-18): 0.22 (MeOH).

RMN-¹H (500 MHz, CDCl₃), δ (ppm): 7.60 (m, 2 H, 2 NHCO), 6.50 (m, 2 H, J=6.5 Hz, 2 N^{α}HCO), 5.36 (m, 4 H, 2 CH^{9,10}), 4.85 (m, 2 H, 2 NHBOC), 4.48 (m, J=5.7 Hz, 2 H, 2 CH^{α}), 3.38 (m, J=13.2 Hz, J=6.6 Hz, 2 H, 2 CHₐ^{1′}), 3.28 (m, 4 H, 2 CH_{b}^{1′}, 2 CHₐ^{δ}), 3.11 (m, 2 H, J=13.2 Hz, J=6.3 Hz, 2 CH_{b}^{δ}), 2.48 (m, 12 H, 2 CH₂^{3′}, 4 CH₂^{4′}), 2.23 (t, 4 H, J=7.8 Hz, 2 CH₂²), 2.03 (m, 8 H, 2 CH₂^{8,11}), 1.87-1.40 (m, 34 H, 2 CH₂^{β}, 2 CH₂^{2′}, 2 CH₂³, 2 CH₂^{γ}, 2 C(CH₃)₃), 1.30-1.22 (m, 40 H, 2×10 CH₂^{o1}), 0.91 (t, 6 H, J=6.4 Hz, 2 CH₃¹⁸).

RMN-¹³C (125 MHz, CDCl₃), δ (ppm): 173.2, 171.5, 156.4, 130.0, 129.8, 79.2, 57.0, 53.3, 52.2, 39.7, 39.2, 36.7, 31.9, 30.6, 29.8, 29.5, 29.3, 29.2, 28.5, 27.2, 26.5, 25.7, 25.2, 22.7, 14.1. HRMS (+ESI): C₅₈H₁₁₃N₈O₄ [M+H], calcd.: m/z 1157.9615, found: m/z=1157.9531.

### Example 34 (GSC170 Orn)

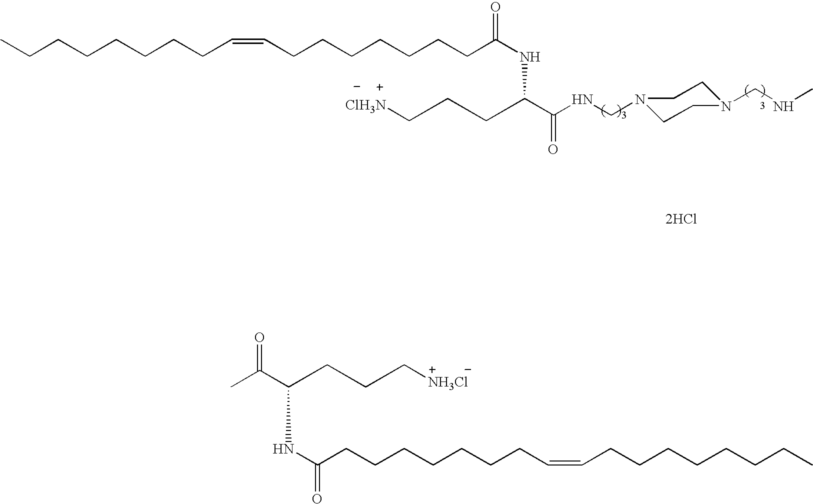

To a solution of compound 5 (138 mg, 0.12 mmol) in EtOAc (10 mL) was added 4.9 N HCl-EtOAc (10 ml). The reaction was stirred at RT for 2 h. The precipitate, was filtrated and washed with ether to give a white solid. Yield 116 mg (0.10 mmol, 83%), Rf (SiO₂): 0.61 (MeOH—NH₄OH 80:20).

RMN-¹H (500 MHz, CDCl₃), δ (ppm): 8.24 (s broad, 2 H, 2 NHCO), 8.04 (d, 2 H, J=7.8 Hz, 2 NH^{α}CO), 7.94 (s broad, 6 H, 2 NH₃⁺), 5.33 (m, 4 H, 2 CH^{9,10}), 4.20 (m, J=5.5 Hz, 2 H, 2 CH^{α}), 3.76 (m 4 H, 4 CHₐₓᵢₐₗ^{4′}), 3.48 (m, 4 H, 4 CH_{ecuat}^{4′}), 3.12 (m, 8 H, 2 CH₂^{1′}, 2 CH₂^{3′}), 2.76 (m, 4 H, J=5.7 Hz, 2 CH₂^{δ}), 2.14 (t, 4 H, J=7.4 Hz, 2 CH₂²), 1.98 (m, 8 H, 2 CH₂^{8,11}), 1.84 (m, 4 H, 2 CH₂^{2′}), 1.70 (m, 2 H, 2 CHₐ^{β}), 1.64-1.40 (m, 10 H, 2 CH_{b}^{β}, 2 CH₂³, 2 CH₂^{γ}), 1.24 (m, 40 H, 2×10 CH₂^{o1}), 0.86 (t, 6 H, J=6.7 Hz, 2 CH₃¹⁸).

RMN-¹³C (125 MHz, CDCl₃), δ (ppm): 172.5, 171.8, 129.7, 79.3, 53.8, 51.9, 48.1, 38.3, 35.3, 31.4, 29.3, 29.2, 28.9, 28.8, 28.7, 26.7, 25.3, 22.2, 14.1. HRMS (+ESI): C₅₈H₁₁₃N₈O₄ [M+H], calcd.: m/z 957.8572, found: m/z=957.8575.

### Example 35 (Compound 7)

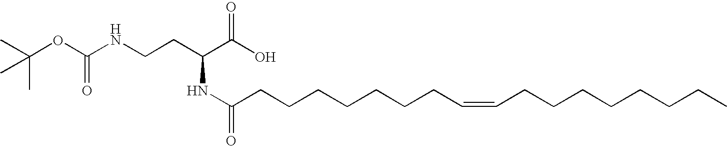

To a solution of N-γ-(t-butoxycarbonyl)-L-diaminobutyric acid (203 mg, 0.93 mmol) in THF (27 mL) were added successively a solution of K₂CO₃ (141 mg, 1.02 mmol) in water (4 mL) and oleoyl succinimidate (371 mg, 0.98 mmol). The reaction was stirred at RT for 16 h and the most of THF was evaporated. Water and CHCl₃ (10 mL each) were added and the mixture was acidified with 1 M HCl to pH: 2. The organic layer was separated, and the aqueous layer was extracted with CHCl₃ (2×10 ml). The organic layer was washed with brine (8 mL), dried (Na₂SO₄), filtrated and evaporated to give a syrup. Yield: 441 mg (0.91 mmol 98%). Rf (SiO₂): 0.35 (CHCl₃—MeOH 85:15).

### Example 36 (Compound 8)

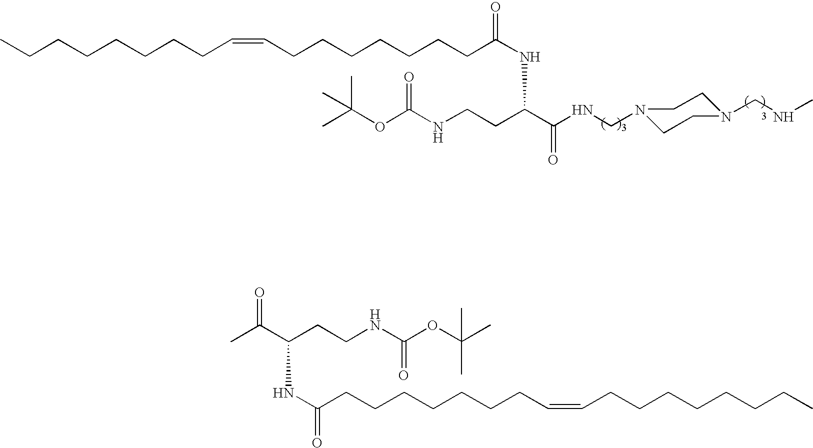

To a solution of aminoacid 7 (410 mg, 0.85 mmol) in CH₂Cl₂ (4 ml) were added benzotriazole-1-yl-oxi-tris-pyrrolidino-phosphonium hexafluorphosphate (442 mg, 0.85 mmol), DIEA (296 μl, 1.70 mmol) and 1,4-Bis(3-aminopropyl)piperazine (67 μl, 0.33 mmol). The mixture was stirred at RT for 16 h. The solvent was removed, CHCl₃ (40 mL) was added and the organic layer was washed with 5% NaHCO₃ (3×12 mL) and brine (30 mL), dried Na₂SO₄), filtrated and evaporated. The residue was purified by column chromatography on reverse phase (C-18) to give a syrup. Yield: 316 mg (0.28 mmol, 85%), Rf (SiO₂): 0.51 (CHCl₃—MeOH 85:15), Rf (C-18): 0.14 (MeOH).

### Example 37 (GSC170 Dab)

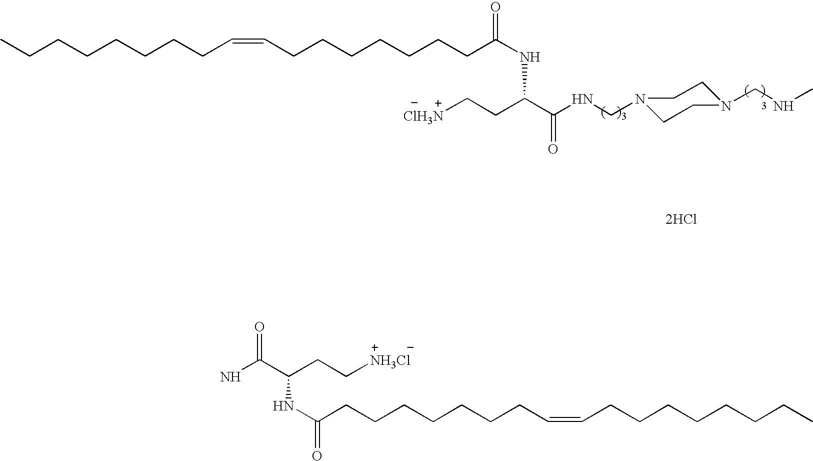

To a solution of compound 8 (244 mg, 0.22 mmol) in EtOAc (15 mL) was added 4.9 N HCl-EtOAc (15 ml). The reaction was stirred at RT for 2 h. The precipitate was filtrated and washed with ether to give a white solid. Yield 198 mg (0.18 mmol, 82%), Rf (SiO₂): 0.52 (MeOH—NH₄OH 85:15).

### Example 38 (Compound 10)

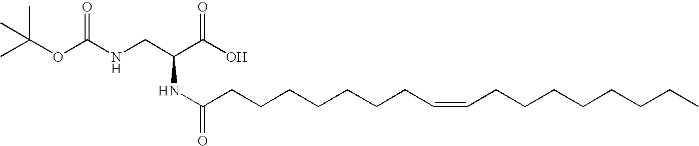

To a solution of N-β-(t-butoxycarbonyl)-L-diaminopropionic acid (560 mg, 2.74 mmol) in THF (77 mL) were added successively a solution of K₂CO₃ (416 mg, 3.02 mmol) in water (11 mL) and oleoyl succinimidate (1.04 g, 2.74 mmol). The reaction was stirred at RT for 18 h and the most of THF was evaporated. Water and CHCl₃ (30 mL each) were added and the mixture was acidified with 1 M HCl to pH: 2. The organic layer was separated, and the aqueous layer was extracted with CHCl₃ (2×30 ml). The organic layer was washed with brine (20 mL), dried (Na₂SO₄), filtrated and evaporated to give a white solid. Yield: 1.25 g (2.67 mmol, 97%). Rf(SiO₂): 0.35 (CHCl₃—MeOH 85:15).

### Example 39 (Compound 11)

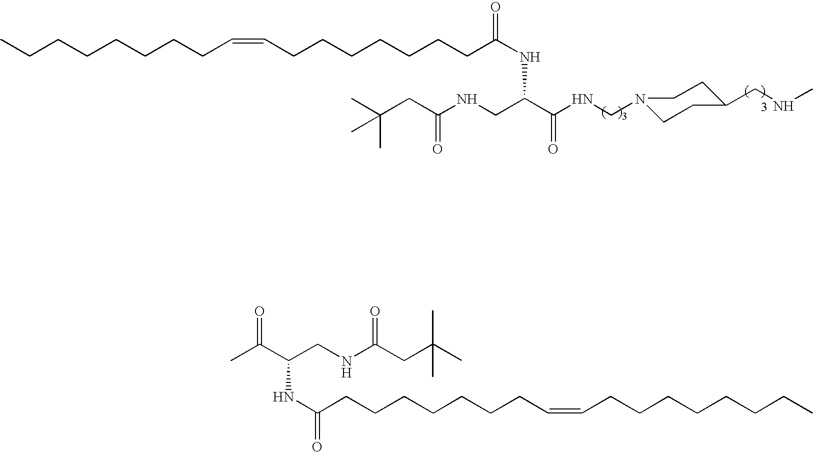

To a solution of aninoacid 10 (1.22 g, 2.60 mmol) in CH₂Cl₂ (14 ml) were added benzotriazole-1-yl-oxi-tris-pyrrolidino-phosphonium hexafluorphosphate (1.35 g, 2.60 mmol), DIEA (910 μl, 5.21 mmol) and 1,4-Bis(3-aminopropyl)piperazine (206 μl, 1.00 mmol). The mixture was stirred at RT for 15 h. The solvent was removed, CHCl₃ (75 mL) was added and the organic layer was washed with 5% NaHCO₃ (3×30 mL) and brine (60 mL), dried (Na₂SO₄), filtrated and evaporated. The residue was purified by column chromatography on reverse phase (C-18) to give a syrup. Yield: 940 mg (0.85 mmol, 85%), Rf(SiO₂): 0.52 (CHCl₃—MeOH 85:15), Rf(C-18): 0.16 (MeOH).

### Example 40 (GSC170 Dap)

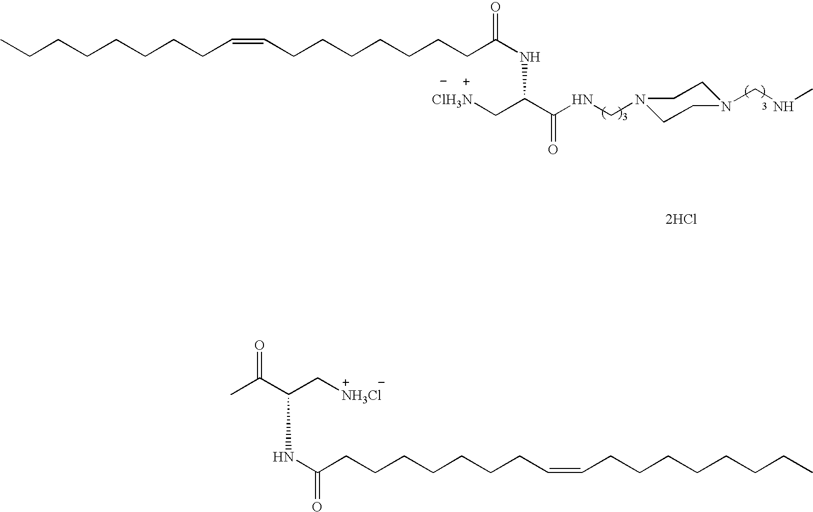

To a solution of compound 11 (355 mg, 0.32 mmol) in EtOAc (30 mL) was added 4.9 N HCl-EtOAc (30 ml). The reaction was stirred at RT for 2 h. The precipitate was filtrated and washed with ether to give a white solid. Yield 280 mg (0.27 mmol, 84%), Rf (SiO₂): 0.34 (MeOH—NH₄OH 99:1).

### Example 41

### Transfection of Recombinant Plasmid Expressing Luciferase into Cells using Lysine-Polyamine-Based Gemini Compounds

Transfection of recombinant plasmid expressing luciferase into cells using lysine-polyamine-based gemini compounds. Transfection studies were performed using the adherent cell line CHO-DG44. Complete medium consisted of MEM alpha medium supplemented with 10% v/v foetal bovine serum and 1× L-Glutamine. All media and supplements were obtained from Life Technologies. In Vitro Gene Transfection. Cells were seeded into Biocat poly-D-lysine 96-well black plates (BD) 16-18 hours prior to transfection at an approximate density of 3×104 cells per well. For transfection, 0.1 mg of the luciferase reporter gene plasmid, pGL3-Control Vector (Promega) per well, was incubated with various concentrations of the diaminoacid-polyamine:peptide-based gemini compounds and complexing agents in a final volume of 100 μl. After 30 minutes incubation at RT, OPTI-MEM® medium (Life Technologies) was added to the transfection mixture and the solution placed on the cells (final volume per well: 100 μl). Following a 3 hour or over night incubation at 37° C., the transfection solution was replaced with complete medium and the cells incubated further at 37° C. Reporter gene assays were performed according to the manufacturer's guidelines (Roche Diagnostics) approximately 48 hours post transfection. Luminescence was measured in a Packard TopCount NXT Microplate Scintillation and Luminescence Counter. FIG. 4. Transfection of CHO-DG44 cells with Gemini surfactant GSC102. The numbers along the x-axis refer to concentration of gemini compounds in mM. The block of 5 bars at the right of the chart shows the data obtained when DNA was premixed with poly-lysine. The block of 5 bars at the left side shows data when no poly-lysine is used. The figures on the Y-axis represent CPS (count per second) from the luciferase assay. Bars represent the mean CPS of 4 experiments±the standard error of the mean. FIG. 5. Transfection of CHO-DG44 cells with Gemini surfactant GSN14. Bars represent the mean CPS (counts per second) of 4 experiments±the standard error of the mean. FIG. 6. Transfection of CHO-DG44 cells with Gemini surfactant GSC197. Bars represent the mean CPS (counts per second) of 4 experiments±the standard error of the mean.

### Example 42

### Delivery of Fluorescent Oligonucleotides to Cell Lines/Primary Cells using Gemini Surfactant 170 (GSC170)

GSC170 (1 mg/ml in water) was diluted to a 10× solution with Optimem serum free media. A FITC-tagged oligonucleotide was similarly diluted in Optimem at 10× final concentration. The GSC170 and oligonucleotide were then mixed 1:1 and incubated for fifteen minutes at room temperature. The adherent cell lines: RBL-2H3, J774 and 16HBE14o were plated out the day before transfection. Murine primary T cells were transfected either inactivated or after differentiation into T helper 2 cells. GSC170:oligo complexes were diluted to 1× in Optimem and added to adherent cells that had been washed once in Optimem then all media removed. Nuclear delivery of the oligonucleotide was oserved over a period of 24 hours and compared to the commercial reagent, Lipofectamine 2000 (LF2K).

Transfection efficiencies are shown in Table 1:

TABLE 1
Transfection efficiencies using GSC170

| | GSC170 (% nuclear) | LF2K (% nuclear) |
|---|---|---|
| RBL-2H3 | 50% | # |
| J774 | 50% | 50% |
| 16HBE14o | 50% | 30% |
| Primary inactivated T cells | 60% | # |
| Activated T helper 2 cells | 60% | # |

# = too low to estimate

All publications, including but not limited to patents and patent applications, cited in this specification are herein incorporated by reference as if each individual publication were specifically and individually indicated to be incorporated by reference herein as though fully set forth.

### BRIEF DESCRIPTION OF THE DRAWINGS

FIG. 1. General scheme for synthesis of diaminoacid-polyamine:peptide based gemini compounds wherein the hydrophobic tail is linked to the α-amino group of a diaminoacid further linked to a polyamine moiety by amide bonds.

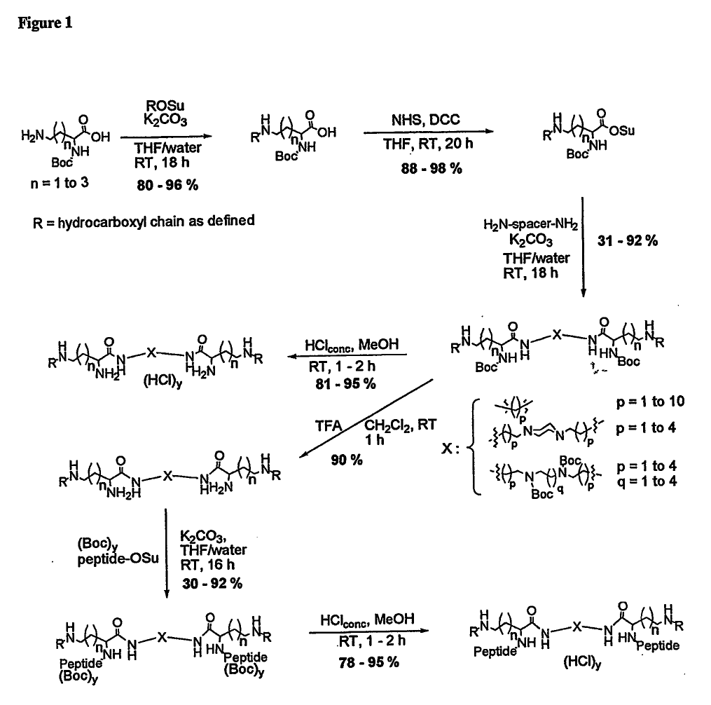

FIG. 2. General scheme for synthesis of diaminoacid-polyamine:peptide based gemini compounds wherein the hydrophobic tail is linked to the terminalamino group of a diaminoacid further linked to a polyamine moiety by amide bonds.

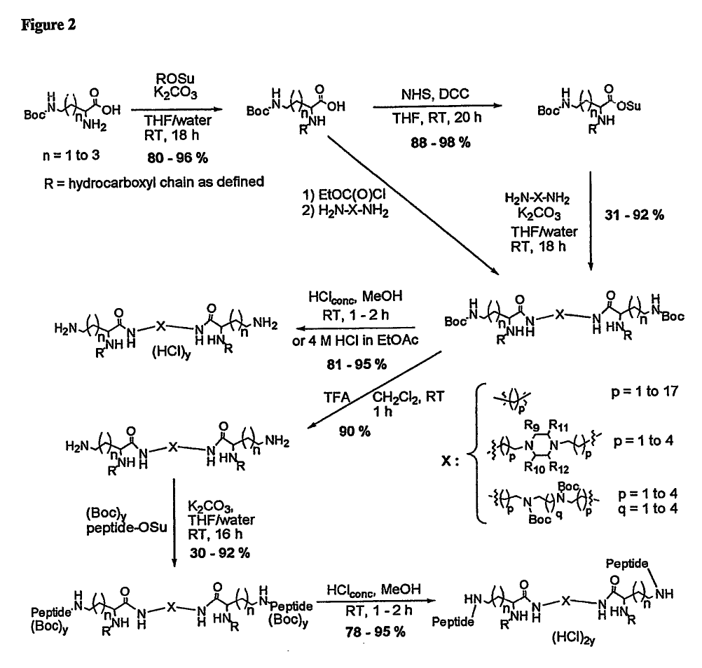

FIG. 3. General scheme for the synthesis of diaminoacid-aminoacid-polyamine:peptide based gemini compounds wherein an aminoacid is linked by an amide bond to the α-amino group of a diaminoacid further linked to a polyamine moiety by amide bonds.

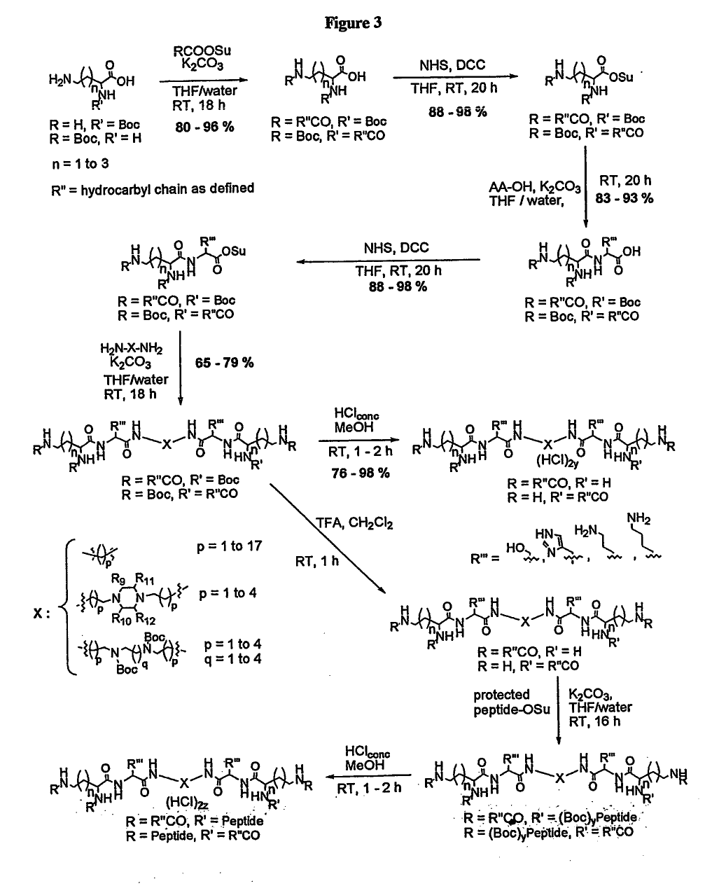

FIG. 4. Transfection of recombinant plasmid expressing luciferase into CHO-DG44 cells using GSC102. The numbers along the x-axis refer to concentration of the gemini compound in mM. The block of 5 bars at the right of the chart shows the data obtained when DNA was premixed with poly-lysine. The block of 5 bars at the left side shows data when no poly-lysine is used. The figures on the Y-axis represent CPS (count per second) from the luciferase assay. Bars represent the mean CPS of 4 experiments±the standard error of the mean.

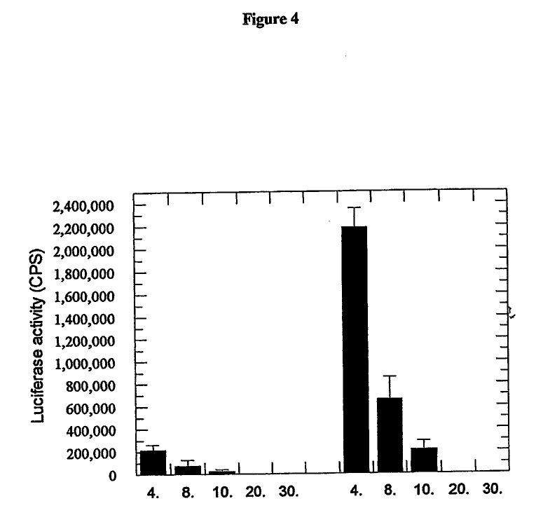

FIG. 5. Transfection of recombinant plasmid expressing luciferase into CHO-DG44 cells using GSN14. The numbers along the x-axis refer to concentration of the gemini compound in mM. The block of 5 bars at the right of the chart shows the data obtained when DNA was premixed with poly-lysine. The block of 5 bars at the left side shows data when no poly-lysine is used. The figures on the Y-axis represent CPS (count per second) from the luciferase assay. Bars represent the mean CPS of 4 experiments±the standard error of the mean.

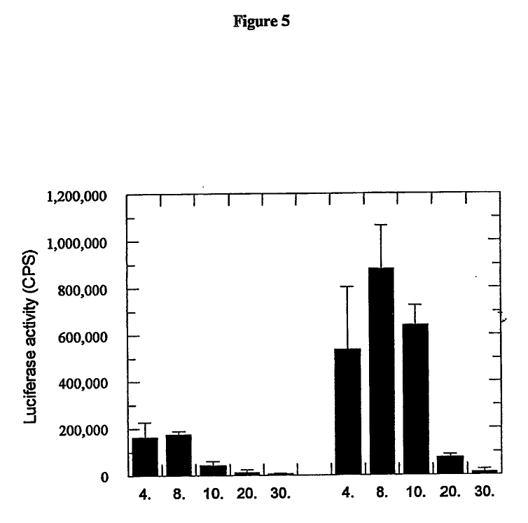

FIG. 6. Transfection of recombinant plasmid expressing luciferase into CHO-DG44 cells using GSC197. The numbers along the x-axis refer to concentration of the gemini compound in mM. The block of 5 bars at the right of the chart shows the data obtained when DNA was premixed with poly-lysine. The block of 5 bars at the left side shows data when no poly-lysine is used. The figures on the Y-axis represent CPS (count per second) from the luciferase assay. Bars represent the mean CPS of 4 experiments±the standard error of the mean.

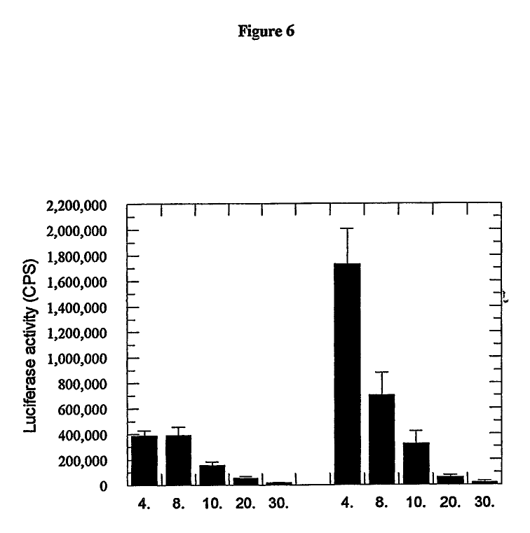

1. A diaminoacid-polyamine:peptide based gemini compound having a diaminoacid-polyamine or a diaminoacid-aminoacid-polyamine backbone and conforming to the general structure of formula (I): 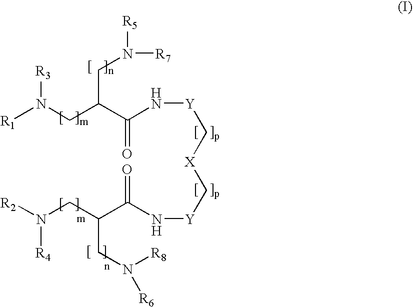
where:
m=0 to 6;
n=0 to 7;
p=0 to 6; and where
X=a bond, CH₂, (CH₂)₂, NH(CH₂)qNH where q=2 to 6, or 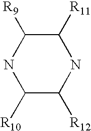
where R₉ to R₁₂, which can be the same or different, are selected from H and CᵣH₂ᵣ₊₁, where r=1 to 6; and where Y=a bond, CH₂, 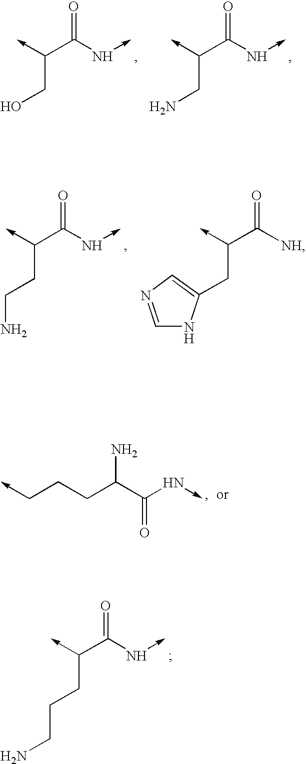
and where R₃, R₄, R₅, R₆, R₇ and R₈ are hydrogen and R₁ and R₂ are saturated or unsaturated hydrocarboxyl groups having up to 24 carbon atoms and linked to the diaminoacid-polyamine backbone by an amide bond;
or
where R₃, R₄, R₅ and R₆ are hydrogen, R₁ and R₂ are saturated or unsaturated hydrocarboxyl groups having up to 24 carbon atoms and linked to the diaminoacid-polyamine backbone by an amide bond, and where R₇ and R₈, which may be the same or different, are peptide groups linked to the diaminoacid-polyamine backbone by amide bonds, in a linear or branched manner, wherein the peptide groups are represented by the general formula (II): 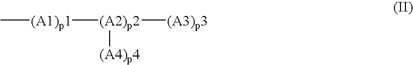
where the values for p1 and p2, which may be the same or different, are from 1 to 5;
and the values for p3 and p4, which may be the same or different, are from 0 to 5;
A1, A3 and A4, which may be the same or different, is an amino acid selected from the group consisting of serine, lysine, ornithine, threonine, histidine, cysteine, arginine and tyrosine; and
A2 is an amino acid selected from the group consisting of lysine, ornithine and histidine; or
a pharmaceutically acceptable salt thereof.

2. A compound according to claim 1 wherein R₁ and R₂ are the same as each other, R₃ and R₄ are the same as each other, R₅ and R₆ are the same as each other, R₇ and R₈ are the same as each other.

3. A compound according to claim 1 wherein A1 is lysine, serine or threonine, and A3 and A4 are lysine, ornithine, histidine or arginine.

4. A compound according to claim 1 wherein the hydrocarboxyl group is selected from the group consisting of:
—C(O)(CH₂)₁₀CH₃
—C(O)(CH₂)₁₂CH₃
—C(O)(CH₂)₁₄CH₃
—C(O)(CH₂)₁₆CH₃
—C(O)(CH₂)₁₈CH₃
—C(O)(CH₂)₂₀CH₃
—C(O)(CH₂)₇CH═CH(CH₂)₅CH₃ natural mixture
—C(O)(CH₂)₇CH═CH(CH₂)₇CH₃ natural mixture
—C(O)(CH₂)₇CH═CH(CH₂)₅CH₃ Cis
—C(O)(CH₂)₇CH═CH(CH₂)₇CH₃ Cis
—C(O)(CH₂)₇CH═CH(CH₂)₅CH₃ Trans
—C(O)(CH₂)₇CH═CH(CH₂)₇CH₃ Trans
—C(O)(CH₂)₇CH═CHCH₂CH═CH(CH₂)₄CH₃
—C(O)(CH₂)₇(CH═CHCH₂)₃CH₃
—C(O)(CH₂)₃CH═CH(CH₂CH═CH)₃(CH₂)₄CH₃
—C(O)(CH₂)₇CHCH(CH₂)₇CH₃
—C(O)CHCHOH(CH₂)₂CH₃ and
—C(O)(CH₂)₂₂CH₃.

5. A compound according to claim 1 where m is 0, n is 2 to 4, X is (CH₂) or (CH₂)₂, Y is a bond and p is 0 to 4.

6. A compound according to claim 1 where m is 0, n is 2 to 4, X is NH(CH₂)qNH, where q is 2 to 5, Y is a bond and p is 2 to 5.

7. A compound according to claim 1 where m is 0, n is 2 to 4, X is 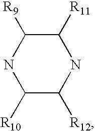
where R₉, R₁₀, R₁₁ and R₁₂ are all H, Y is a bond and p is 2 to 5.

8. A compound according to claim 1 where m is 0, n is 2 to 4, X is (CH₂) or (CH₂)₂, p is 0 to 4 and Y is

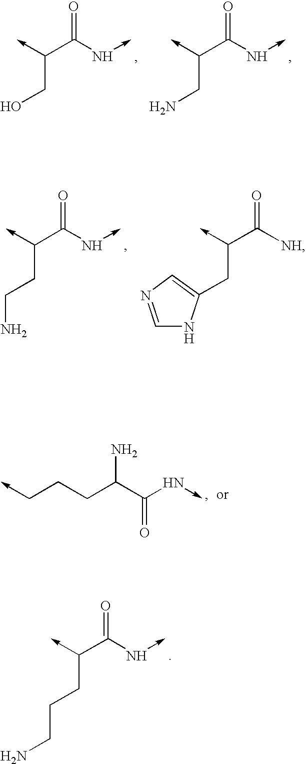

9. A compound according to claim 1 where m is 0, n is 2 to 4, X is NH(CH₂)qNH, where q is 2 to 5, p is 2 to 5 and Y is

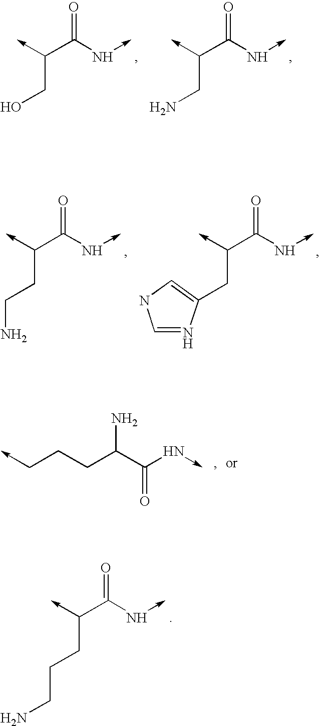

10. A compound according to claim 1 where m is 0, n is 2 to 4, X is 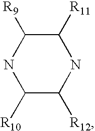
where R₉, R₁₀, R₁₁ and R₁₂ are all H, p is 2 to 5 and Y is

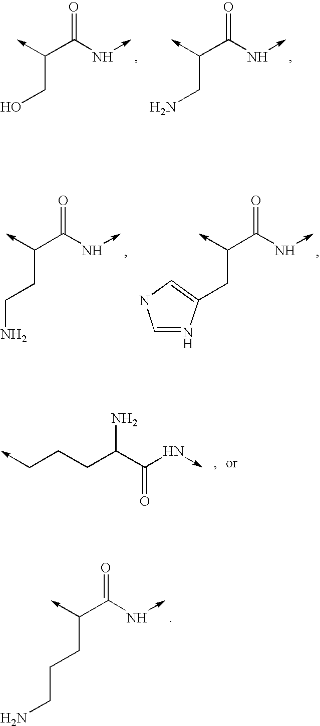

11. A compound according to claim 1 where X is 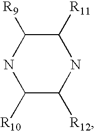
Y is a bond, p is 1 to 6 and n is 1 to 7.

12. A salt of the compound of claim 1 which is represented by the formula:

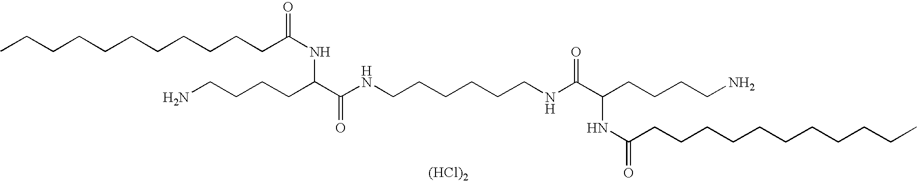

13. A salt of the compound of claim 1 which is represented by the formula:

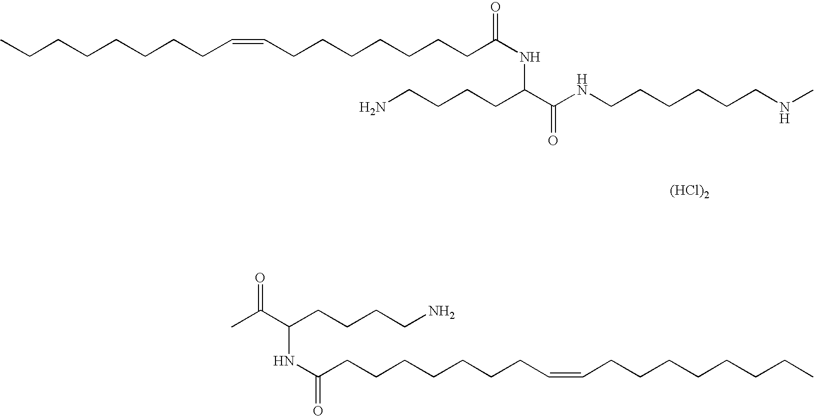

14. A salt of the compound of claim 1 which is represented by the formula:

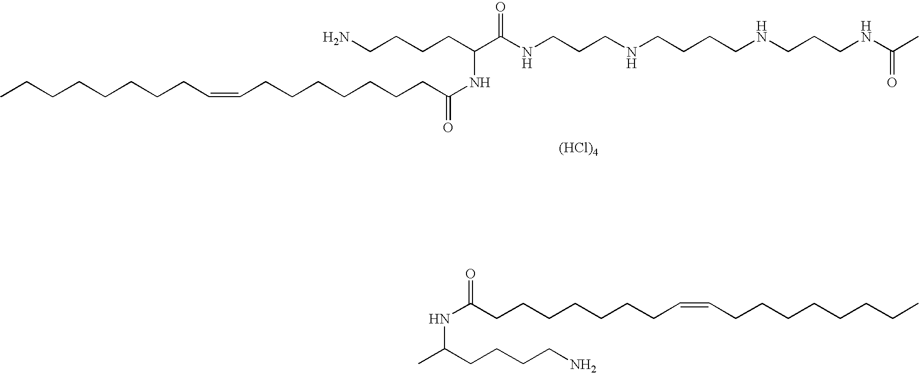

15. A salt of the compound of claim 1 which is represented by the formula:

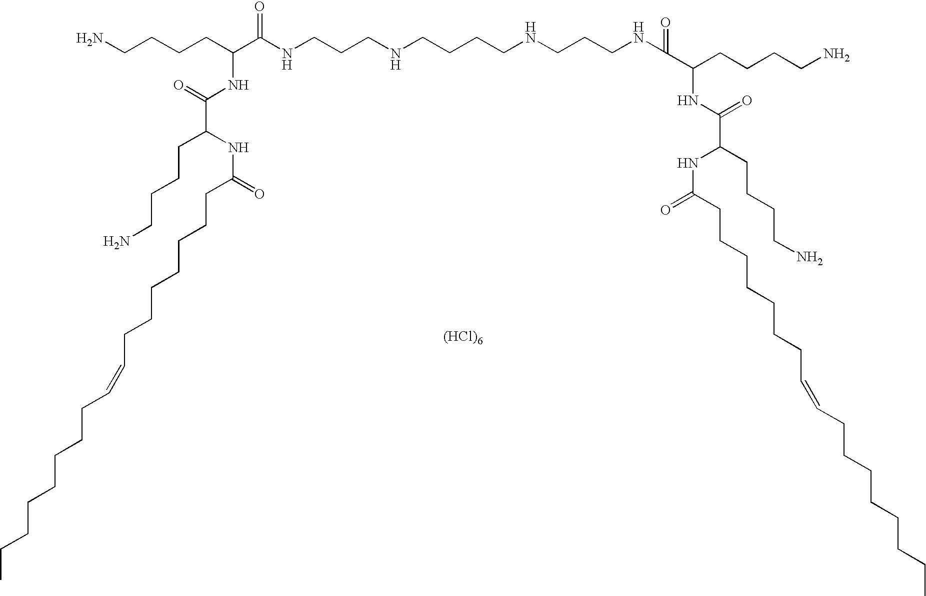

16. A salt of the compound of claim 1 which is represented by the formula:

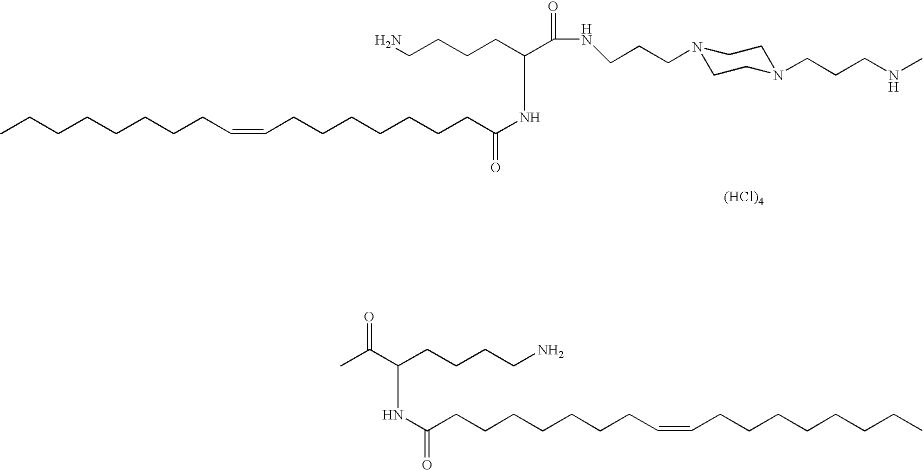

17. A salt of the compound of claim 1 which is represented by the formula:

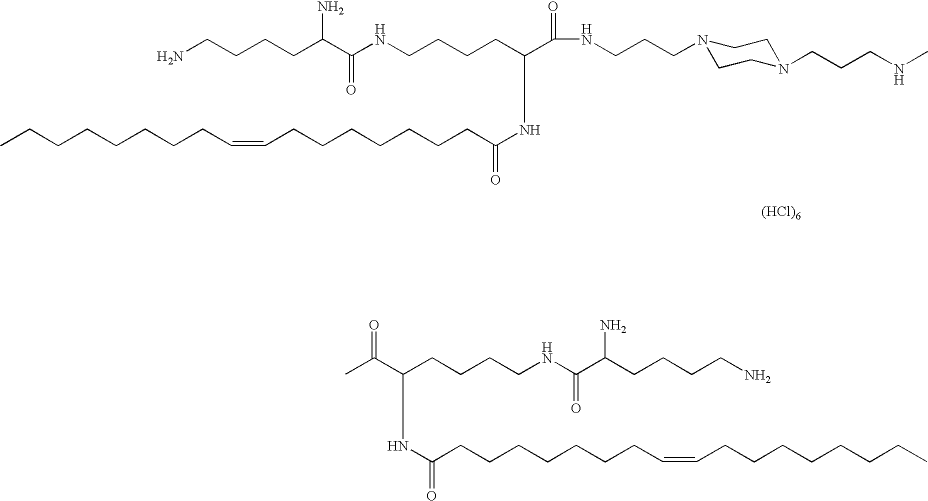

18. A salt of the compound of claim 1 which is represented by the formula:

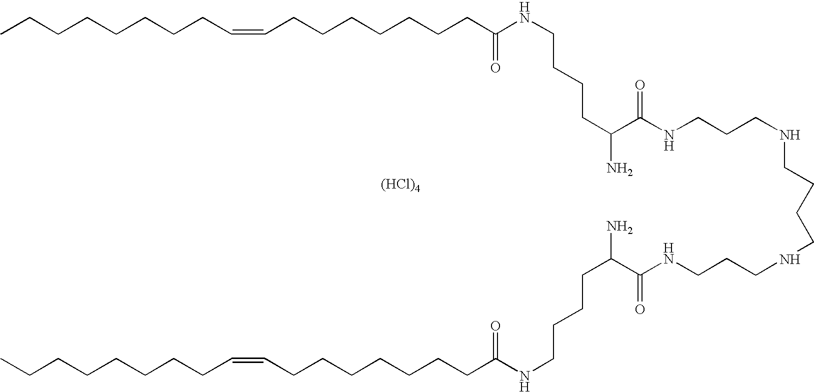

19. (canceled)

20. The method of claim 31 wherein the compound further includes a supplement selected from the group consisting of:
(i) a neutral carrier; or
(ii) a complexing reagent.

21. The method according to claim 20 wherein the neutral carrier is dioleyl phosphatidylethanolamine (DOPE).

22. (canceled)

23. The method according to claim 20 wherein the complexing reagent is a peptide comprising mainly basic amino acids.

24. The method according to claim 23 wherein the peptide consists of basic amino acids.

25. The method according to claim 23 wherein the basic amino acids are selected from the group consisting of lysine, ornithine, and arginine.

26. (canceled)

27. (canceled)

28. (canceled)

29. (canceled)

30. A process for preparing diaminoacid-polyamine-based gemini compounds of claim 1 which process comprises the coupling of a succinimidate ester of a diaminoacid linked to its α or terminal amino group to an hydrocarboxyl chain to a polyamine linker using potassium carbonate as a base in a mixture of tetrahydrofuran and water as solvents.

31. A method of enabling transfection of DNA or RNA or analogs thereof comprising the step of administering an effective amount of a compound of claim 1, or a pharmaceutically acceptable salt thereof, to a culture to facilitate transfer of the DNA or RNA or analogs thereof into a eukaryotic or prokaryotic cell.

32. A method of facilitating transfer of a polynucleotide or anti-infective compound into a prokaryotic or eukariotic organism comprising the step of administering an effective amount of a compound of claim 1 or a pharmaceutically acceptable salt thereof to the organism to treat infection.
